(19) United States
(12) Patent Application Publication
(10) Pub. No.: US 2025/0049900 A1
(43) Pub. Date: Feb. 13, 2025

(54) METHOD OF SAFE ADMINISTRATION OF TAU PHOSPHOPEPTIDE CONJUGATE

(71) Applicants: Janssen Pharmaceuticals, Inc., Titusville, NJ (US); AC Immunce SA, Lausanne (CH)

(72) Inventors: Andrea Pfeifer, St. Légier (CH); Elizabeth Anne Ramsburg, Chalfont, PA (US)

(21) Appl. No.: 18/696,444

(22) PCT Filed: Sep. 29, 2022
(86) PCT No.: PCT/US2022/077279

§ 371 Date: Mar. 28, 2024

Related U.S. Application Data
(60) Provisional application No. 63/261,793, filed on Sep. 29, 2021.

(51) Int. Cl. A61K 39/00; A61K 39/385; A61K 39/39; A61P 25/28; A61P 37/04

(52) U.S. Cl. CPC A61K 39/0007; A61K 39/385; A61K 39/39; A61P 25/28; A61P 37/04; A61K 2039/54; A61K 2039/545; A61K 2039/55505; A61K 2039/55561; A61K 2039/575; A61K 2039/6037; A61K 2039/6068; A61K 2039/6081

(57) ABSTRACT

Methods for inducing anti-phosphorylated Tau antibodies without inducing a serious adverse event in humans are described. The methods include administering to the subject an effective amount of a composition containing a Tau phosphopeptide conjugated to an immunogenic carrier.

### CROSS-REFERENCE TO RELATED APPLICATION

This application is an International Application, which claims priority to U.S. Provisional Patent Application No. 63/261,793, filed Sep. 29, 2021, the disclosure of which is incorporated herein by reference in its entirety.

### REFERENCE TO AN ELECTRONIC SEQUENCE LISTING

The contents of the electronic sequence listing (065794_8WO1.xml; Size: 35,700 bytes; and Date of Creation: Aug. 29, 2022) is herein incorporated by reference in its entirety.

### FIELD OF THE INVENTION

This application is in the field of medicine. The application in particular relates to methods for inducing an immune response against tau protein in a subject suffering from a neurodegenerative disease, disorder or condition with a composition comprising a phosphorylated Tau (pTau) peptide conjugated to an immunogenic carrier.

### BACKGROUND

Alzheimer's Disease (AD) is a progressive debilitating neurodegenerative disease that affects an estimated 44 million people worldwide (Alzheimers.net). AD therapies that are currently commercialized aim to act on the clinical symptoms, but do not target the pathogenic processes that underlie the disease (disease-modifying effect). Unfortunately, the current therapies are only minimally efficacious, and there is therefore an urgent need to develop and test additional preventive and therapeutic measures.

The hallmark pathologies for Alzheimer's Disease are an accumulation of extracellular plaques comprising notably aggregated amyloid beta protein and intracellular “tangles” or aggregations of hyperphosphorylated Tau protein. The molecular events that lead to accumulation of these proteins are poorly characterized. For amyloid, it is hypothesized that aberrant cleavage of the amyloid precursor protein leads to an accumulation of the aggregation-prone fragment comprising amino acids 1-42. For Tau, it is hypothesized that dysregulation of either kinases, phosphatases, or both, leads to aberrant phosphorylation of Tau. Once Tau becomes hyperphosphorylated it loses the ability to effectively bind and stabilize microtubules, and instead accumulates in the cytoplasm of the affected neuron. The unbound and hyperphosphorylated Tau appears to form first oligomers and then higher order aggregates, the presence of which presumably negatively affects the function of the neuron in which they form, perhaps via interruption of normal axonal transport.

In developed nations, individuals diagnosed with Alzheimer's Disease or other dementing Tauopathies are commonly treated with cholinesterase inhibitors (e.g., Aricept®) or memantine (e.g., Namenda™). These drugs, although reasonably well tolerated, have very modest efficacy. For example, Aricept® delays the worsening of symptoms for 6-12 months in approximately 50% of the treated individuals. The remainder of treatment is non-pharmacologic, and focuses on making patients more capable of managing day to day tasks as their cognitive ability declines.

Immunotherapies are currently under development for the prevention and treatment of AD. Active immunization with an antigen related to AD can potentially stimulate a response of both antibody-based and cellular immunity against AD. However, evaluation of the first widely tested human anti-amyloid beta vaccine was stopped in 2002. Meningoencephalitis, a type of central nervous system inflammation that can be fatal, was observed in clinical studies in AD patients of the active immunotherapeutic agent AN-1792 that targeted Aβ (Orgogozo et al., 2003). The encephalitic reactions, which occurred in 6% of patients exposed to the AN-1792, are thought to have been induced by unwanted AD-specific T-cell activation.

To date few studies have been conducted with agents specifically targeting Tau pathology. Tau immunotherapies are now moving into clinical trials but the field is still in its infancy and mechanistic understanding of the efficacy and safety of the various approaches is not well established (Sigurdsson, Neurodegener Dis. 2016; 16(0): 34-38). Encephalitis, inflammation of the brain, has also been reported in mice immunized against full-length Tau protein. However, no adverse effects were reported from animals immunized with a single injection of phosphorylated-Tau peptide under a CNS proinflammatory milieu (Rosenmann H., 2013. Curr. Alzheimer Res. 10, 217-228).

The long-term safety profile of a non-Tau phosphopeptide vaccine (AADvac1) in human patients with mild to moderate Alzheimer's Disease has been published (Novak et al., Alzheimer's Research & Therapy (2018) 10:108). The vaccine contains a synthetic peptide derived from amino acids 294 to 305 of the Tau sequence coupled to keyhole limpet hemocyanin (KLH) through an N-terminal cysteine. It was administered in doses of 40 μg of the peptide (CKDNIKHVPGGGS, SEQ ID NO: 13) coupled to KLH, with aluminium hydroxide adjuvant (containing 0.5 mg A13+) in a phosphate buffer volume of 0.3 ml. The observed adverse events (AEs) from the 26 patients enrolled in the study and linked to AADvac1 treatment in the phase 1 study (FUNDAMANT study) were injection site reactions (erythema, swelling, warmth, pruritus, pain, nodule). One or more of these AEs were observed in 50% of patients on AADvac1 treatment. Injection site reactions were reversible and predominantly mild in presentation. Six serious adverse events (SAEs) were observed (abdominal strangulated hernia, dehydration, acute psychosis, behavioral and psychiatric symptoms of dementia, second-degree atrioventricular block, and sinus bradycardia). None of the SAEs were judged by the investigators to be related to AADvac1 treatment. No allergic or anaphylactic reactions were observed. No safety signals emerged in laboratory assessment (coagulation, blood biochemistry, hematology, and urinalysis), vital sign assessment, or neurological and physical examination. No safety signals were detected by MRI assessment. No oedematous changes occurred. No meningeal changes and no meningoencephalitis were observed. New micro-hemorrhages were observed in one ApoE4 homozygote, and superficial hemosiderin was detected in one ApoE4 heterozygote, both events were clinically silent. This was considered to be consistent with the background incidence of such lesions in the AD patient population.

However, the safety profile of a Tau phosphopeptide conjugate in human patients has not been reported. There is a need for a safe and effective treatment for neuronal degenerative disease, such as Alzheimer's Disease.

### SUMMARY OF THE INVENTION

In one general aspect, the application relates to a method of inducing antibodies against Tau, preferably at least one of phosphorylated Tau and enriched paired helical filaments (ePHFs), in a human subject in need thereof, the method comprising administering to the human subject a composition comprising 5 μg to 200 μg per dose of a conjugate having the structure of formula (I):

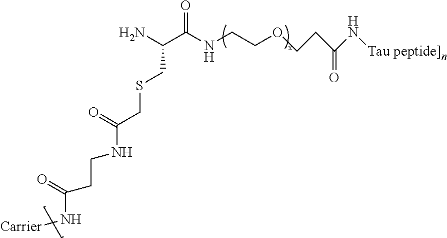

or having the structure of formula (II):

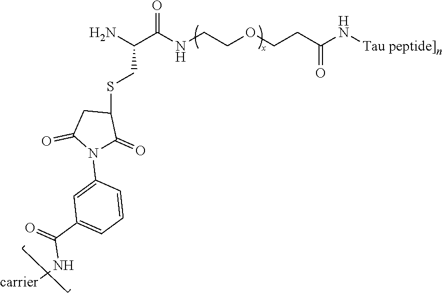

wherein
x is an integer of 0 to 10, preferably 2 to 6, most preferably 3;
n is an integer of 3 to 15, preferably 3 to 12;
Carrier represents an immunogenic carrier selected from the group consisting of keyhole limpet hemocyanin (KLH), tetanus toxoid, CRM197 and an outer membrane protein mixture from N. meningitidis (OMP), or a derivative thereof; and
Tau peptide represents a Tau phosphopeptide having an amino acid sequence selected from the group consisting of SEQ ID NO: 1 to SEQ ID NO: 3 and SEQ ID NO: 5 to SEQ ID NO: 12.

In one general aspect, the application relates to a composition for inducing antibodies against Tau, preferably at least one of phosphorylated Tau and enriched paired helical filaments (ePHFs), in a human subject in need thereof, the composition comprises 5 μg to 200 μg per dose of a conjugate having the structure of formula (I):

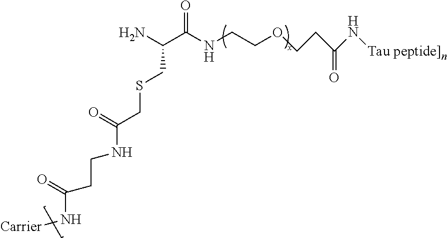

or having the structure of formula (II):

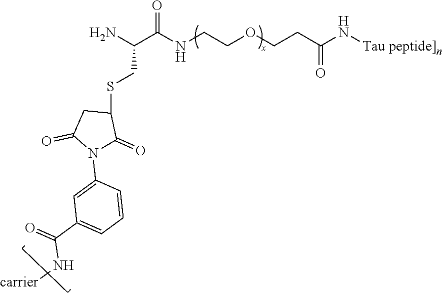

wherein
x is an integer of 0 to 10, preferably 2 to 6, most preferably 3;
n is an integer of 3 to 15, preferably 3 to 12;
Carrier represents an immunogenic carrier selected from the group consisting of keyhole limpet hemocyanin (KLH), tetanus toxoid, CRM197 and an outer membrane protein mixture from N. meningitidis (OMP), or a derivative thereof; and
Tau peptide represents a Tau phosphopeptide having an amino acid sequence selected from the group consisting of SEQ ID NO: 1 to SEQ ID NO: 3 and SEQ ID NO: 5 to SEQ ID NO: 12.

In one embodiment, the composition further comprises a pharmaceutically acceptable carrier.

In one embodiment of the application, the conjugate comprises a tau phosphopeptide having an amino acid sequence selected from the group consisting of SEQ ID NO: 1 to SEQ ID NO: 3 or SEQ ID NO: 5 to SEQ ID NO: 12 conjugated to CRM197 via a linker.

Preferably, the Tau peptide is the Tau phosphopeptide having the amino acid sequence of SEQ ID NO: 2. More than one tau phosphopeptide, such as 2, 3, 4, 5, 6, 7, 8, 9, 10 or more tau phosphopeptides, can be conjugated to one carrier protein. More preferably, the conjugate has the structure of:

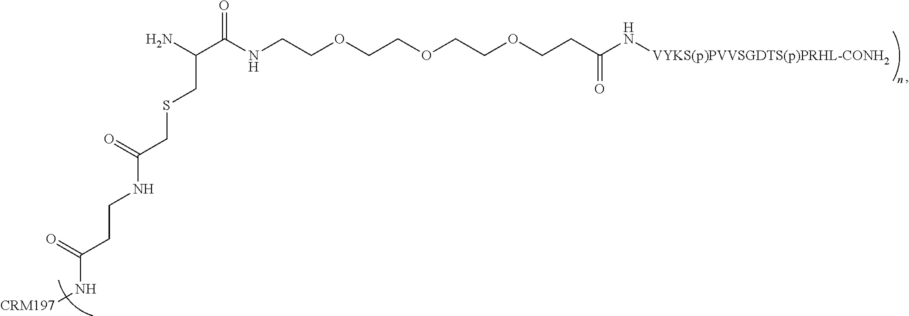

wherein n is an integer of 3 to 7 and VYKS(p)PVVSGDTS(p)PRHL-CONH₂ comprises the phospho-tau peptide of SEQ ID NO:2.

According to embodiments of the application, the composition further comprises at least one adjuvant. For example, the at least one adjuvant can comprise a TLR9 agonist, such as a CpG oligonucleotide having a nucleotide sequence selected from the group consisting of SEQ ID NO: 14 to SEQ ID NO: 18. In one embodiment, the composition further comprises a CpG oligonucleotide having the nucleotide sequence of SEQ ID NO: 14. In another embodiment, the composition further comprises aluminum hydroxide. In yet another embodiment, the composition further comprises a CpG oligonucleotide having a nucleotide sequence selected from the group consisting of SEQ ID NO: 14 to SEQ ID NO: 18, and aluminum hydroxide.

According to an embodiment of the application, a method of inducing antibodies against Tau, preferably at least one of phosphorylated Tau and paired helical filaments (PHFs), in a human subject in need thereof, comprises administering to the human subject a composition comprising a pharmaceutically acceptable carrier, aluminum hydroxide, a CpG oligonucleotide having the nucleotide sequence of SEQ ID NO: 14, and 5 μg to 200 μg per dose of a conjugate having the structure of:

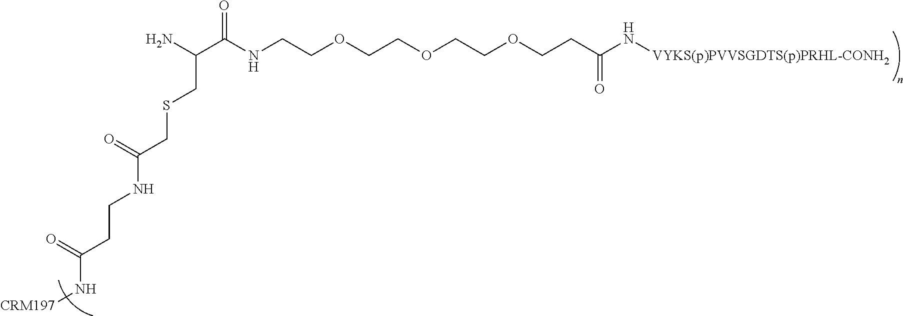

wherein n is an integer of 3 to 7 and VYKS(p)PVVSGDTS(p)PRHL-CONH₂ comprises the phospho-tau peptide of SEQ ID NO:2.

In certain embodiments, a method of the application comprises administering to the human subject a composition comprising 5 μg, 10 μg, 15 μg, 20 μg, 25 μg, 30 μg, 35 μg, 40 μg, 45 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, or any value in between, per dose of the conjugate described herein.

In certain embodiments, the composition is administered intramuscularly. In other embodiments, the composition is administered subcutaneously.

In certain embodiments, the antibodies comprise IgG antibodies against the phosphorylated Tau (pTau), preferably having an anti-pTau IgG titer at least 50, 60, 70, 80, 90, 100 or more times higher than that of a placebo control.

In certain embodiments, the antibodies comprise IgG antibodies against non-phosphorylated Tau, preferably having an anti-Tau IgG titer at least 50, 60, 70, 80, 90, 100 or more times higher than that of a placebo control.

In certain embodiments, the antibodies comprise IgG antibodies against an enriched Paired Helical Filament (ePHF), preferably having an anti-ePHF IgG titer at least 2, 3, 4, 5, 6, 7, 8, 9, 10, 11, 12, 13, 14, 15, 16, 17, 18, 19, 20 or more times higher than that of a placebo control.

In certain embodiments, a method of the application further comprises administering to 17 the subject a second dose of the composition comprising a pharmaceutically acceptable carrier and 5 μg to 200 μg, such as 15 μg or 60 μg, per dose of the conjugate 4 to 12 weeks, such as 8 weeks, after the initial administration of the composition.

In certain embodiments, the administration of the second dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the second dose of the composition.

In certain embodiments, a method of the application further comprises administering to the subject a third dose of the composition comprising a pharmaceutically acceptable carrier and 5 μg to 200 μg, such as 15 μg or 60 μg, per dose of the conjugate 20 to 28 weeks, such as 24 weeks, after the initial administration of the composition.

In certain embodiments, the administration of the third dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the third dose of the composition.

In certain embodiments, a method of the application further comprises administering to the subject a fourth dose of the composition comprising a pharmaceutically acceptable carrier and 5 μg to 200 μg, such as 15 μg or 60 μg, per dose of the conjugate 44 to 52 weeks, such as 48 weeks, after the initial administration of the composition.

In certain embodiments, the fourth dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the fourth dose of the composition.

In one general aspect, the application relates to a method of inducing a sustained immune response against a phosphorylated Tau protein (pTau) in a human subject in need thereof, comprising:
i. intramuscularly administering to the subject a primer vaccine comprising an effective amount of a conjugate; and
ii. intramuscularly administering to the subject a first booster vaccine comprising the effective amount of the conjugate 6-10 weeks after the administration of the primer vaccine,
   wherein:
the sustained immune response lasts at least about 20 weeks after the administration of the primer vaccine;
the conjugate has the structure of formula (I):

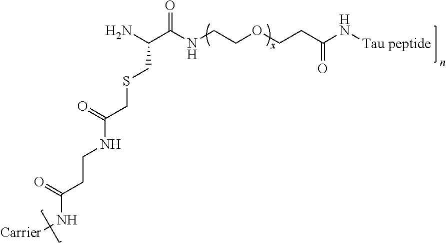

or has the structure of formula (II):

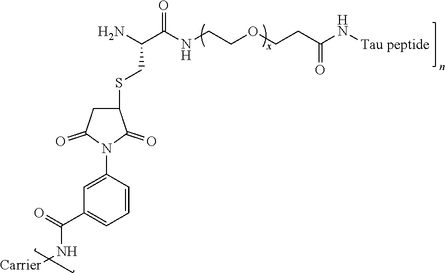

wherein
   x is an integer of 0 to 10, preferably 2 to 6, most preferably 3;
   n is an integer of 3 to 15, preferably 3 to 12;
   Carrier represents an immunogenic carrier selected from the group consisting of keyhole limpet hemocyanin (KLH), tetanus toxoid, CRM197 and an outer membrane protein mixture from N. meningitidis (OMP), or a derivative thereof; and
   Tau peptide represents a Tau phosphopeptide having an amino acid sequence selected from the group consisting of SEQ ID NO: 1 to SEQ ID NO: 3 and SEQ ID NO: 5 to SEQ ID NO: 12;
the effective amount of the conjugate comprises 5 μg to 200 μg per dose of the conjugate.

In certain embodiments, the Carrier is CRM197.

In certain embodiments, the effective amount of the conjugate comprises 15 μg per dose of the conjugate.

In certain embodiments, the effective amount of the conjugate comprises 60 μg per dose of the conjugate.

In certain embodiments, a method of the application further comprises intramuscularly administering to the subject a second booster vaccine composition comprising the effective amount of the conjugate 20 to 26 weeks after the administration of the primer vaccine, and the sustained immune response lasts at least about 36 weeks after the administration of the primer vaccine.

In certain embodiments, the second booster vaccine composition is administered 24 weeks after the administration of the primer vaccine, and the sustained immune response lasts at least about 48 weeks after the administration of the primer vaccine.

In certain embodiments, a method of the application further comprises intramuscularly administering to the subject a third booster vaccine composition comprising the effective amount of the conjugate 45-50 weeks after the administration of the primer vaccine, and the sustained immune response lasts at least about 67 weeks after the administration of the primer vaccine.

In certain embodiments, the third booster vaccine composition is administered 48 weeks after the administration of the primer vaccine, and the sustained immune response lasts at least about 74 weeks after the administration of the primer vaccine.

In certain embodiments, the sustained immune response comprises an IgG response against phosphorylated Tau (pTau), preferably having an anti-pTau IgG titer at least 50, 60, 70, 80, 90, 100 or more times higher than that of a placebo control.

In certain embodiments, the sustained immune response comprises an IgG response against non-phosphorylated Tau, preferably having an anti-Tau IgG titer at least 50, 60, 70, 80, 90, 100 or more times higher than that of a placebo control.

In certain embodiments, the sustained immune response comprises an IgG response against enriched Paired Helical Filament (ePHF), preferably having an anti-ePHF IgG titer at least 2, 3, 4, 5, 6, 7, 8, 9, 10, 11, 12, 13, 14, 15, 16, 17, 18, 19, 20 or more times higher than that of a placebo control.

In certain embodiments, the subject is in need of clearance of aggregates of Tau.

In certain embodiments, the subject is in need of a treatment of a neurodegenerative disease or disorder caused by or associated with the formation of neurofibrillary lesions. Preferably, the human subject is in need of a treatment of Alzheimer's Disease, such as early Alzheimer's Disease, mild cognitive impairment (MCI) due to Alzheimer's Disease, mild Alzheimer's Disease, or mild to moderate Alzheimer's Disease. In certain embodiments, the subject is amyloid positive in the brain but does not yet show significant cognitive impairment. In other embodiments, the subject has abnormal level of cerebrospinal fluid (CSF) Abeta amyloid 42 (Aß42) consistent with AD pathology. In another embodiment, the subject is in need of a treatment of a neurodegenerative disease or disorder caused by or associated with the formation of neurofibrillary lesions.

Further aspects, features and advantages of the present invention will be better appreciated upon a reading of the following detailed description of the invention and claims.

### BRIEF DESCRIPTION OF THE DRAWINGS

The foregoing summary and the following detailed description of the invention will be better understood when read in conjunction with the appended drawings. For the purpose of illustrating the invention, there are shown in the drawings embodiments which are presently preferred. It should be understood, however, that the invention is not limited to the precise embodiments shown in the drawings.

FIG. 1 is a graph of the geometric mean (±95% Confidence Interval) of the anti-pTau IgG response directed against a phosphorylated Tau peptide (pTau) over time in sub-cohort 2.1 following treatment with either JACI-35.054 (15 μg or 60 μg) or placebo (ITT analysis set).

FIG. 2 is a graph of the geometric mean (±95% Confidence Interval) of the anti-Tau IgG response directed against a non-phosphorylated Tau peptide over time in sub-cohort 2.1 following treatment with either JACI-35.054 (15 μg or 60 μg) or placebo (ITT analysis set).

FIG. 3 is a graph of the geometric mean (±95% Confidence Interval) of anti-ePHF (enriched Paired Helical Filaments) IgG titers over time in sub-cohort 2.1 following treatment with either JACI-35.054 (15 μg or 60 μg) or placebo (ITT analysis set).

FIG. 4 is a graph of the epitope recognition profile of antibodies in 8 AD patients induced by vaccination with JACI-35.054 (15 μg) as determined by epitope mapping ELISA on short 8-mer overlapping peptides, covering phospho-peptides T3.30 (SEQ ID NO: 19) and T3.85 (SEQ ID NO: 21) and non-phospho-peptides T3.56 (SEQ ID NO: 20) and T3.86 (SEQ ID NO: 22). FIG. 4A shows the epitope recognition profile of anti-phosphorylated Tau antibodies in sub-cohort 2.1. FIG. 4B shows the epitope recognition profile of anti-Tau antibodies in sub-cohort 2.1. O.D.=optical density.

### DETAILED DESCRIPTION OF THE INVENTION

Various publications, articles, patents and patent applications are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive “or” and not to an exclusive “or”. For example, a condition 1 or 2 is satisfied by any one of the following: 1 is true (or present) and 2 is false (or not present), 1 is false (or not present) and 2 is true (or present), and both 1 and 2 are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The invention provides a method of inducing antibodies against Tau, preferably at least one of phosphorylated Tau and enriched paired helical filaments (ePHFs), without inducing a serious adverse event, such as encephalitis, in a human subject in need thereof. In particular embodiments, the method comprises administering to the subject an effective amount of a conjugate comprising a Tau phosphopeptide covalently linked to an immunogenic carrier either directly or via a linker.

As used herein, the term “anti-phosphorylated Tau antibody” refers to an antibody that binds to Tau that has been phosphorylated on an amino acid residue at one or more locations of the amino acid sequence of Tau. The phosphorylated amino acid residues can be, e.g., serine (Ser), threonine (Thr) or tyrosine (Tyr). The site on phosphorylated Tau to which the anti-phosphorylated Tau antibody binds is preferably a site that is specifically phosphorylated in neurodegenerative diseases such as Alzheimer's Disease. Examples of sites of phosphorylated Tau to which the anti-phosphorylated Tau antibody binds include, for example, Tyr18, Ser199, Ser202, Thr205, Thr212, Ser214, Ser396, Ser404, Ser409, Ser422, Thr427. As used throughout the present application, the amino acid positions are given in reference to the sequence of human microtubule-associated protein tau isoform 2 having the amino acid sequence represented in GenBank Accession No. NP_005901.2.

The ability to induce anti-phosphorylated Tau antibodies upon administration can be determined by testing a biological sample (e.g., blood, plasma, serum, PBMCs, urine, saliva, feces, Instersitial Fluid (ISF), CSF or lymph fluid) from the subject for the presence of antibodies, e.g., IgG or IgM antibodies, directed to the immunogenic Tau peptide(s) administered in the pharmaceutical composition (see, for example, Harlow, 1989, Antibodies, Cold Spring Harbor Press). For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT or Antibody-Dependent Cellular Phagocytosis (ADCP) Assay.

As used herein, the term “adverse event” (AE) refers to any untoward medical occurrence in a patient administered a pharmaceutical product and which does not necessarily have a causal relationship with the treatment. According to embodiments of the invention, AEs are rated on a 3-point scale of increasing severity using the following definitions: mild (grade 1), referring to an AE that is easily tolerated by the subject, which causes minimal discomfort and does not interfere with everyday activities; moderate (grade 2), referring to an AE that is sufficiently discomforting to interfere with normal everyday activities and intervention may be needed; severe (grade 3), referring to an AE that prevents normal everyday activities, and treatment or other intervention is usually needed. A serious AE (SAE) can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; in patient hospitalization, e.g., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that may jeopardize the patients or may require medical or surgical intervention to prevent one of the other outcomes listed above (e.g., intensive treatment in an emergency room or at home for allergic bronchospasm or blood dyscrasias or convulsions that do not result in hospitalization). Hospitalization is official admission to a hospital. Hospitalization or prolongation of a hospitalization constitutes criteria for an AE to be serious; however, it is not in itself considered an SAE. In the absence of an AE, hospitalization or prolongation of hospitalization should not be reported as a SAE by the participating investigator. This can be the case, in the following situations: the hospitalization or prolongation of hospitalization is needed for a procedure required by the protocol; or the hospitalization or prolongation of hospitalization is a part of a routine procedure followed by the center (e.g., stent removal after surgery). This should be recorded in the study file. Hospitalization for elective treatment of a pre-existing condition that did not worsen during the study is not considered an AE.

Complications that occur during hospitalization are AEs. If a complication prolongs hospitalization, or meets any of the other SAE criteria, then the event is an SAE.

As used herein, the term “encephalitis” refers to an inflammation of the brain which can result from infectious and non-infectious causes. As used herein, the term “meningoencephalitis” refers to a condition characterized by infection or inflammation of the brain meninges and of the brain. The diagnosis of encephalitis or meningoencephalitis can be determined by techniques known to those skilled in the art in view of the present disclosure, for example, by clinical, neurological and psychiatric examinations, biological sampling including blood and CSF samplings, MRI scanning and electroencephalography (EEG).

As used herein, the term “Tau” or “Tau protein”, also known as microtubule-associated protein Tau, MAPT, neurofibrillary tangle protein, paired helical filament-Tau, PHF-Tau, MAPTL, MTBT1, refers to an abundant central and peripheral nervous system protein having multiple isoforms. In the human central nervous system (CNS), six major Tau isoforms ranging in size from 352 to 441 amino acids in length exist due to alternative splicing (Hanger et al., Trends Mol Med. 15:112-9, 2009). Examples of Tau include, but are not limited to, Tau isoforms in the CNS, such as the 441-amino acid longest Tau isoform (4R2N), also named microtubule-associated protein tau isoform 2, that has four repeats and two inserts, such as the human Tau isoform 2 having the amino acid sequence represented in GenBank Accession No. NP_005901.2. Other examples of Tau include the 352-amino acid long shortest (fetal) isoform (3R0N), also named microtubule-associated protein tau isoform 4, that has three repeats and no inserts, such as the human Tau isoform 4 having the amino acid sequence represented in GenBank Accession No. NP_058525.1. Examples of Tau also include the “big Tau” isoform expressed in peripheral nerves that contains 300 additional residues (exon 4a). Friedhoff et al., Biochimica et Biophysica Acta 1502 (2000) 122-132. Examples of Tau include a human big Tau that is a 758 amino acid-long protein encoded by an mRNA transcript 6762 nucleotides long (NM_016835.4), or isoforms thereof. The amino acid sequence of the exemplified human big Tau is represented in GenBank Accession No. NP_058519.3. As used herein, the term “Tau” includes homologs of Tau from species other than human, such as Macaca Fascicularis (cynomolgus monkey), rhesus monkeys or Pan troglodytes (chimpanzee). As used herein, the term “Tau” includes proteins comprising mutations, e.g., point mutations, fragments, insertions, deletions and splice variants of full-length wild type Tau. The term “Tau” also encompasses post-translational modifications of the Tau amino acid sequence. Post-translational modifications include, but are not limited to, phosphorylation.

As used herein, the term “peptide” or “polypeptide” refers to a polymer composed of amino acid residues, related naturally occurring structural variants, and synthetic non-naturally occurring analogs thereof linked via peptide bonds. The term refers to a peptide of any size, structure, or function. Typically, a peptide is at least three amino acids long. A peptide can be naturally occurring, recombinant, or synthetic, or any combination thereof. Synthetic peptides can be synthesized, for example, using an automated polypeptide synthesizer. Examples of Tau peptides include any peptide of Tau protein of about 5 to about 30 amino acids in length, preferably of about 10 to about 25 amino acids in length, more preferably of about 16 to about 21 amino acids in length. In the present disclosure, peptides are listed from N to C terminus using the standard three or one letter amino acid abbreviation, wherein phosphoresidues are indicated with “p.” Examples of Tau peptides useful in the invention include, but are not limited to, Tau peptides comprising the amino acid sequence of any of SEQ ID NOs: 1-12, or Tau peptides having an amino acid sequence that is at least 75%, 80%, 85%, 90% or 95% identical to the amino acid sequence of any of SEQ ID NOs: 1-12.

The avidity of an antibody can be measured by avidity index using methods known in the art in view of the present disclosure. The titers of antibodies against a particular antigen are measured at two different concentrations of the coated antigen: one is the saturated concentration, where all antibodies can bind to the antigen and another one is at a low concentration, where only antibodies with the highest binding capacity can bind to the antigen. As used herein, “avidity index” refers to the ratio of the levels of antibody titers measured at the low- and the high-density coating of the antigen. For example, avidity of antibodies against an antigen, such as ePHF or pTau, can be measured at different time points after an immunization or following different immunizations, to evaluate whether the avidity (as measured by the avidity index) increases over time. As used herein, antibodies with an “increased avidity” or “increased binding avidity” to an antigen refers to antibodies with an increased avidity index to the antigen over time during the course of a treatment or immunization. An increased avidity suggests a potential affinity maturation of the antibodies.

As used herein, the term “phosphopeptide” or “phospho-epitope” refers to a peptide that is phosphorylated at one or more amino acid residues. Examples of Tau phosphopeptides include any Tau peptide comprising one or more phosphorylated amino acid residues. Any suitable tau phosphopeptides known to those skilled in the art can be used in the conjugate in view of the present disclosure. According to particular embodiments, the one or more Tau phosphopeptides comprise the amino acid sequence of one of SEQ ID NOs: 1-3 or 5-12, or an amino acid sequence that is at least 75%, 80%, 85%, 90% or 95% identical to the amino acid sequence of one of SEQ ID NOs: 1-3 or 5-12, wherein one or more of the indicated amino acid residues are phosphorylated. Preferably, the Tau phosphopeptide comprises the amino acid sequence of one of SEQ ID NOs: 1-3. Abnormal phosphorylated Tau aggregates readily into insoluble oligomers which are neurotoxic and contribute to neurodegeneration (Goedert et al, 1991). The oligomers progress to tangles of so-called paired helical filaments (PHF) (Alonso et al., 2001). The degree of neurofibrillary tangle pathology has been consistently shown to be correlated to the degree of dementia in AD subjects (Bierer et al, 1995; Braak and Braak, 1991; Delacourte, 2001).

The Tau peptides useful for the present invention can be synthesized by solid phase peptide synthesis or by recombinant expression systems. Automatic peptide synthesizers are commercially available from numerous suppliers, such as Applied Biosystems (Foster City, Calif.). Recombinant expression systems can include bacteria, such as E. coli, yeast, insect cells, or mammalian cells. Procedures for recombinant expression are described by Sambrook et al., Molecular Cloning: A Laboratory Manual (C.S.H.P. Press, NY 2d ed., 1989).

### Conjugate

Examples of conjugates useful for the present invention include, but are not limited to, the Tau phosphopeptide conjugate described in U.S. patent publication No. US 2019/0119341, the disclose of which is herein incorporated by reference in its entirety.

According to particular aspects, the conjugate has the following structure:

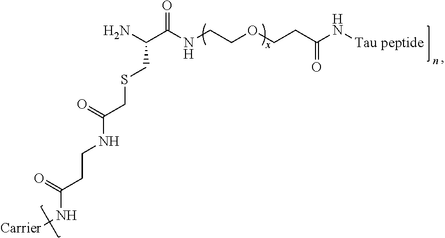

or the structure of formula (II):

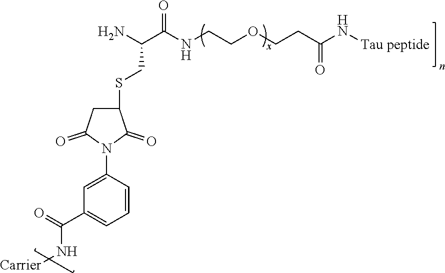

wherein
x is an integer of 0 to 10;
n is an integer of 2 to 15, preferably 3-11;
Carrier represents an immunogenic carrier; and
Tau peptide represents a tau phosphopeptide.

As used herein, the term “immunogenic carrier” refers to an immunogenic substance that can be coupled to a tau peptide. An immunogenic moiety coupled to a tau peptide can induce an immune response and elicit the production of antibodies that can specifically bind the tau peptide. Immunogenic moieties are operative moieties that include proteins, polypeptides, glycoproteins, complex polysaccharides, particles, nucleic acids, polynucleotides, and the like that are recognized as foreign and thereby elicit an immunologic response from the host. Any suitable immunogenic carrier known to those skilled in the art in view of the present disclosure can be used in the invention. According to particular embodiments, the immunogenic carrier is keyhole limpet hemocyanin (KLH), tetanus toxoid, CRM197 (a non-toxic form of diphtheria toxin), an outer membrane protein mixture from N. meningitidis (OMP), or a derivative thereof. According to particular embodiments, the immunogenic carrier is CRM197.

According to particular embodiments, the tau peptide is conjugated to the immunogenic carrier via a linker. As used herein, the term “linker” refers to a chemical moiety that joins a immunogenic carrier to a tau peptide. Any suitable linker known to those skilled in the art in view of the present disclosure can be used in the invention. The linkers can be, for example, a single covalent bond, a substituted or unsubstituted alkyl, a substituted or unsubstituted heteroalkyl moiety, a polyethylene glycol (PEG) linker, a peptide linker, a sugar-based linker, or a cleavable linker, such as a disulfide linkage or a protease cleavage site, or an amino acid, or a combination thereof. Examples of the linker can comprises one or more of polyethylene glycol (PEG), succinimidyl 3-(bromoacetamido)propionate (SBAP), m-maleimidobenzoyl-N-hydroxysuccinimide ester (MBS), or one or more amino acids such as Cys, Lys or sometimes Ser or Thr, or a combination thereof.

According to particular embodiments, x is an integer of 1 to 10, 2 to 9, 2 to 8, 2 to 7, 2 to 6, 2 to 5, 2 to 4, or 2 to 3. According to particular embodiments, x is 3.

According to particular embodiments, multiple tau phosphopeptides can be conjugated to one immunogenic carrier. In some embodiments, n is 2 to 15, 3 to 11, 3 to 9, 3 to 8, or 3 to 7.

According to particular embodiments, the conjugate comprises one or more tau peptides. According to particular embodiments, the tau peptides of the conjugate can be the same or different.

According to particular embodiments, the tau phosphopeptide consists of the amino acid sequence of one of SEQ ID NOs: 1-3.

According to particular embodiments, the linker comprises (C2H40)x—cysteine—acetamidopropionamide or m-maleimidobenzoyl-N-hydroxysuccinimide ester—cysteine—(C2H4O)x, wherein x is an integer of 0 to 10, such as 0, 1, 2, 3, 4, 5, 6, 7, 8, 9, or 10.

According to particular embodiments, the carrier is covalently linked to the N-terminus of the tau phosphopeptide, via a linker.

According to other particular embodiments, the carrier is covalently linked to the C-terminus of the tau peptide, via a linker.

According to particular embodiments, the conjugate has the structure of:

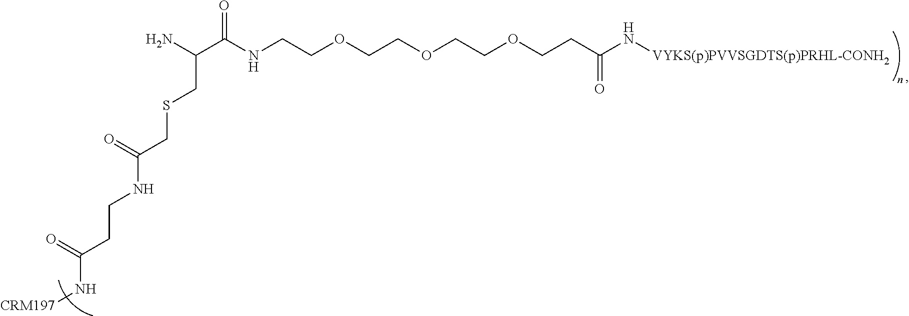

wherein n is an integer of 2 to 15, preferably 3-11, more preferably 3-7 and VYKS(p)PVVSGDTS(p)PRHL-CONH₂ comprises the phospho-tau peptide of SEQ ID NO:2.

Conjugates of the invention can be made by methods known in the art in view of the present disclosure. For example, the above conjugate can be formed by reacting succinimidyl-3-(bromoacetamido)propionate (SBAP):

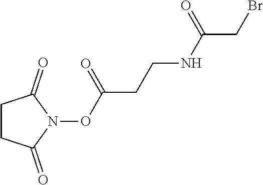 [SMILES: O=C(CBr)NCCC(=O)ON1C(=O)CCC1=O]

with an amino group of CRM197 to form an amide linkage. This CRM197 precursor can be subsequently reacted with the tau peptide (e.g., the Tau phosphopeptide of SEQ ID NO: 2) conjugated at its N-terminus or at its C-terminus to a PEG-cysteine linker with a free nucleophilic thiol group to form the tau phosphopeptide conjugate.

### Pharmaceutical Compositions

Pharmaceutical compositions comprising an effective amount of a conjugate useful for the invention, together with a pharmaceutically acceptable excipient and/or carrier can be made using methods known in the art in view of the present disclosure. The optimal ratios of each component in the compositions can be determined by techniques well known to those skilled in the art in view of the present disclosure.

Pharmaceutically acceptable excipients and/or carriers are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like. It will be understood that the characteristics of the carrier, excipient or diluent will depend on the route of administration for a particular application.

The pharmaceutical composition can contain a mixture of conjugates with the same immunogenic tau peptide. Alternatively, the pharmaceutical composition can contain a mixture of conjugates with different immunogenic tau peptides of the present invention.

According to particular embodiments, a conjugate can be administered in combination with a suitable adjuvant to achieve the desired immune response in the subject. Suitable adjuvants can be administered before, after, or concurrent with administration of conjugate of the present invention. Preferred adjuvants augment the intrinsic response to an immunogen without causing conformational changes in the immunogen that affect the qualitative form of the response.

In one embodiment, adjuvants useful for a method of the application are the aluminum salts (alum), such as aluminum hydroxide, aluminum phosphate, and aluminum sulfate.

In another embodiment, adjuvants useful for a method of the application are TLR agonists, such as CpG oligonucleotides. As used herein, the term “CpG oligonucleotide”, “CpG oligodeoxynucleotide” or “CpG ODN” refers to an oligonucleotide comprising at least one CpG motif. As used herein, “oligonucleotide,” “oligodeoxynucleotide” or “ODN” refers to a polynucleotide formed from a plurality of linked nucleotide units. Such oligonucleotides can be obtained from existing nucleic acid sources or can be produced by synthetic methods. As used herein, the term “CpG motif” refers to a nucleotide sequence which contains unmethylated cytosine-phosphate-guanine (CpG) dinucleotides (i.e., a cytosine (C) followed by a guanine (G)) linked by a phosphate bond or a phosphodiester backbone or other internucleotide linkages, such as phosphorothioate (ps), phosphorodithioate (ps2), methylphosphonate (mp), or methylphosphorothioate (rp). Phosphorothioate, phosphorodithioate, methylphosphonate and methylphosphorothioate are stabilizing internucleotide linkages, while phosphodiester is a naturally-occurring internucleotide linkage. Oligonucleotide phosphorothioates are typically synthesized as a random racemic mixture of Rp and Sp phosphorothioate linkages. Any suitable CpG oligonucleotide known to those skilled in the art can be used in the invention in view of the present disclosure. Examples of such CpG oligonucleotides include, but are not limited to CpG2006 (also known as CpG 7909), CpG 1018, CpG2395, CpG2216 or CpG2336.

According to particular embodiments, the CpG oligonucleotide is lipidated, i.e., conjugated (covalently linked) to a lipid moiety. As used herein, a “lipid moiety” refers to a moiety containing a lipophilic structure. Lipid moieties, such as an alkyl group, a fatty acid, a triglyceride, diglyceride, steroid, sphingolipid, glycolipid or a phospholipid, particularly a sterol such as cholesterol, or fatty acids, when attached to highly hydrophilic molecules, such as nucleic acids, can substantially enhance plasma protein binding and consequently circulation half-life of the hydrophilic molecules. In addition, binding to certain plasma proteins, such as lipoproteins, has been shown to increase uptake in specific tissues expressing the corresponding lipoprotein receptors (e.g., LDL-receptor HDL-receptor or the scavenger receptor SR-B1). In particular, a lipid moiety conjugated to the phosphopeptides and/or CpG oligonucleotide allows anchoring the said peptides and/or oligonucleotides into the membrane of a liposome via a hydrophobic moiety.

Such adjuvants can be used with or without other specific immunostimulating agents, such as MPLA Class (3 De-O-acylated monophosphoryl lipid A (MPL™), monophosphoryl hexa-acyl Lipid A 3-deacyl synthetic (3D-(6-acyl) PHAD®), PHAD™, PHAD®-504, 3D-PHAD®) lipid A), polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 90/14837), containing 5% Squalene, 0.5% Tween 80, and 0.5% Span 85 (optionally containing various amounts of MTP-PE) formulated into submicron particles using a microfluidizer; SAF, containing 10% Squalene, 0.4% Tween 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) 0.2% Tween 80, and one or more bacterial cell wall components selected from the group consisting of monophosphoryl lipid A (MPL™), trehalose dimycolate (TDM), and cell wall skeleton (CWS), preferably MPL™+CWS (Detox™). Other adjuvants include Complete Freund's Adjuvant (CFA), and cytokines, such as interleukins (IL-1, IL-2, and IL-12), macrophage colony stimulating factor (M-CSF), and tumor necrosis factor (TNF).

In certain embodiments, a pharmaceutical composition useful for a method of the application further comprises one or more suitable adjuvants described herein, such as an aluminum salt, e.g., aluminum hydroxide, aluminum phosphate, and/or aluminum sulfate, and/or a CpG, e.g., CpG2006 (also known as CpG 7909), CpG 1018, CpG2395, CpG2216 or CpG2336. In one embodiment, the pharmaceutical composition comprises a pharmaceutically acceptable carrier, aluminum hydroxide, CpG 7909 and a conjugate of a Tau phosphopeptide covalently linked to CRM197 via a linker.

In other embodiments, a pharmaceutical composition comprises a conjugate described herein, one or more adjuvant, and a buffer comprising one or more amino acids, such as histidine or glycine, one or more carbohydrates, such as glucose or sucrose, and/or a surfactant, such as polysorbate 80, polysorbate 20, etc.

### Methods of Use

A general aspect of the application relates to a method of safely inducing an immune response against Tau protein in a human subject suffering from a neurodegenerative disease, disorder, or condition, comprising administering to the subject a pharmaceutical composition comprising an effective amount of a phosphorytlated Tau conjugate. According to particular aspects, the immune response is induced against Tau protein, preferably phosphorylated Tau protein, more preferably ePHF.

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease, and should be decided according to the judgment of the practitioner and each patient's circumstances. For example, the effective amount of Tau phosphopeptide conjugated to an immunogenic carrier protein also depends on whether adjuvant is also administered, with higher dosages being required in the absence of adjuvant. Effective doses can be extrapolated from dose-response curves derived from in vitro or animal model test systems.

Because one or more Tau phosphopeptides can be conjugated to an immunogenic carrier, the effective amount of the conjugate includes the total weight of the immunogenic carrier protein, the one or more Tau phosphopeptides conjugated therto, and the one or more linkers (if used) in the conjugate. According to embodiments of the application, the effective amount of the conjugate is from about 5 μg to about 200 μg per dose, preferably about 15 μg to about 150 μg, of the immunogenic carrier per dose, such as 5 μg, 10 μg, 15 μg, 20 μg, 25 μg, 30 μg, 35 μg, 40 μg, 45 μg, 50 μg, 60 μg, 70 μg, 80 μg, 90 μg, 100 μg, 110 μg, 120 μg, 130 μg, 140 μg, 150 μg, 175 μg, 200 μg, or any value in between, per dose. Preferably, the effective does is 15 μg, up to 60 μg, such as 45 μg, 50 μg, 55 μg, 60 μg, or any value in between, or up to 150 μg, such as 120 μg, 125 μg, 130 μg, 135 μg, 140 μg, 145 μg, 150 μg, or any value in between, per dose.

As used herein, the terms “induce” and “stimulate” and variations thereof refer to any measurable increase in cellular activity. Induction of an immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

The ability to induce or stimulate an anti-Tau immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by methods readily known in the art, e.g., by measurement of cytokine profiles secreted by activated effector cells including those derived from CD4+ and CD8+ T-cells (e.g., quantification of IL-4 or IFN gamma-producing cells by ELISPOT), by determination of the activation status of immune effector cells (e.g., T-cell proliferation assays by a classical [3H] thymidine uptake), by assaying for antigen-specific T lymphocytes in a sensitized subject (e.g., peptide-specific lysis in a cytotoxicity assay, etc.).

The ability to stimulate a cellular and/or a humoral response can be determined by testing a biological sample (e.g., blood, plasma, serum, PBMCs, urine, saliva, feces, CSF or lymph fluid) from the subject for the presence of antibodies directed to the immunogenic tau peptide(s) administered in the pharmaceutical composition (see, for example, Harlow, 1989, Antibodies, Cold Spring Harbor Press). For example, titers of antibodies produced in response to administration of a composition providing an immunogen can be measured by enzyme-linked immunosorbent assay (ELISA), dot blots, SDS-PAGE gels, ELISPOT or Antibody-Dependent Cellular Phagocytosis (ADCP) Assay.

The conjugate composition can be administered by parenteral, topical, intravenous, oral, subcutaneous, intra-arterial, intracranial, intraperitoneal, intradermal, intranasal, or intramuscular means for prophylactic and/or therapeutic treatment. The most typical route of administration of an immunogenic agent is subcutaneous or intramuscular injection. This latter type of injection is most typically performed in the arm or leg muscles.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 1 week, or 2 weeks, or 3 weeks, or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two years after administration of the priming composition.

According to particular aspects, one or more boosting immunizations can be administered. The antigens in the respective priming and boosting compositions, however many boosting compositions are employed, need not be identical, but should share antigenic determinants or be substantially similar to each other.

As known to those skilled in the art, immunogenicity, boostability and sustainability are important considerations for the effectiveness of a vaccine. It is discovered in the present invention that the administration of an effective amount of a conjugate described herein is able to induce a potent antibody response against pTau in a patient in need thereof, such as a patient in need of treating an Alzheimer's Disease (e.g., mild to moderate Alzheimer's Disease or early Alzheimer's Disease) or mild cognitive impairment (MCI) due to Alzheimer's Disease. The antibody response is sustainable, e.g., lasting at least 6 weeks. The antibody response is also boosted by one or more subsequent boosting administrations. As used herein, “boosted” in the context of an antibody response refers to the antibody response that is maintained or enhanced after a subsequent administration as measured at least two weeks after the administration of the subsequent administration. For example, an antibody response is “boosted” by a subsequent administration, if there is an increase of the antibody titer when measured 2 weeks after the subsequent administration as compared with the antibody titer before the subsequent administration.

According to particular embodiments, the human subject is in need of treatment of a neurodegenerative disease, disorder, or condition.

As used herein a “neurodegenerative disease, disorder, or condition” includes any neurodegenerative disease, disorder, or condition known to those skilled in the art in view of the present disclosure. Examples of neurodegenerative diseases, disorders, or conditions include neurodegenerative diseases or disorders caused by or associated with the formation of neurofibrillary lesions, such as Tau-associated diseases, disorders or conditions, referred to as Tauopathies. According to particular embodiments, the neurodegenerative disease, disorder, or condition includes any of the diseases or disorders which show co-existence of Tau and amyloid pathologies including, but not is limited to, Alzheimer's Disease, Parkinson's Disease, Creutzfeldt-Jacob disease, Dementia pugilistica, Down Syndrome, Gerstmann-Straussler-Scheinker disease, inclusion body myositis, prion protein cerebral amyloid angiopathy, traumatic brain injury, amyotrophic lateral sclerosis, parkinsonism-dementia complex of Guam, Non-Guamanian motor neuron disease with neurofibrillary tangles, argyrophilic grain dementia, corticobasal degeneration, Dementia Lewy Amyotrophic Lateral sclerosis, diffuse neurofibrillary tangles with calcification, frontotemporal dementia, preferably frontotemporal dementia with parkinsonism linked to chromosome 17 (FTDP-17), frontotemporal lobar dementia, Hallervorden-Spatz disease, multiple system atrophy, Niemann-Pick disease type C, Pick's disease, progressive subcortical gliosis, progressive supranuclear palsy, Subacute sclerosing panencephalitis, Tangle only dementia, Postencephalitic Parkinsonism, Myotonic dystrophy, chronic traumatic encephalopathy (CTE), Primary age-related Tauopathy (PART), cerebral angiopathy or Lewy body dementia (LBD). According to particular embodiments, the neurodegenerative disease, disorder, or condition is Alzheimer's Disease or another Tauopathy. According to preferred embodiments, the neurodegenerative disease, disorder, or condition is Alzheimer's Disease.

The clinical course of Alzheimer's Disease can be divided into stages, with progressive patterns of cognitive and functional impairments. The stages can be defined using grading scales known in the art including, e.g., NIA-AA Research Framework (see, e.g., Dubois et al., Alzheimer's & Dementia 12 (2016) 292-323, Dubois et al., Lancet Neurol 2014; 13: 614-29, Jack et al., Alzheimer's & Dementia 14 (2018) 535-562) and the Clinical Demential Rating Scale (CDR) (see, e.g., Berg L. Clinical Dementia Rating (CDR). Psychopharmacol Bull. 1988; 24(4):637-639.), the contents of each of which are hereby incorporated by reference in their entirety.

For example, National Institute on Aging-Alzheimer's Association (NIA-AA) research framework defines AD biologically, by neuropathologic change or biomarkers, and treats cognitive impairment as a symptom/sign of the disease rather than the definition of the disease (see, e.g., Clifford R J, NIA-AA Research Framework: Toward a biological definition of Alzheimer's disease. Alzheimer's & Dementia 14 (2018) 535-562, the content of which is incorporated herein by reference). According to the NIA-AA definition, an individual with biomarker evidence of Aβ deposition alone (abnormal amyloid PET scan or low CSF Aβ42 or Aβ42/Aβ40 ratio) with a normal pathologic tau biomarker would be assigned the label “Alzheimer's pathologic change,” and the term “Alzheimer's Disease” would be applied if both biomarker evidence of Aβ and pathologic tau are present. The NIA-AA also developed a system for staging severity of AD. In particular, under the NIA-AA definition (reproduced from Text Box 2 of Clifford R J, 2018, supra):

### Definition

A: A3 biomarkers determine whether or not an individual is in the Alzheimer's continuum.
T: Pathologic tau biomarkers determine if someone who is in the Alzheimer's continuum has Alzheimer's Disease

### Staging Severity:

(N): Neurodegenerative/neuronal injury biomarkers
(C): Cognitive symptoms
A and T indicate specific neuropathologic changes that define Alzheimer's Disease, whereas (N) and (C) are not specific to Alzheimer's disease and are therefore placed in parentheses.

According to preferred embodiments, the neurodegenerative disease, disorder, or condition is early Alzheimer's Disease, mild cognitive impairment (MCI) due to Alzheimer's Disease or mild Alzheimer's Disease.

In some embodiments, the neurodegenerative disease, disorder, or condition is mild to moderate Alzheimer's Disease.

In some embodiments, the subject in need of a treatment is amyloid positive in the brain but does not yet show significant cognitive impairment. The amyloid deposition in the brain can be detected using methods known in the art, such as PET scan, immunoprecipitation mass spectrometry or other methods (e.g., use of CSF biomarkers) (Clifford R J, NIA-AA Research Framework: Toward a biological definition of Alzheimer's disease. Alzheimer's & Dementia 14 (2018) 535-562).

In other embodiments, the human subject in need of a treatment has abnormal level of CSF Abeta amyloid 42 (Aß42) consistent with AD pathology. For example, the subect can have low leval of CSF Aβ42 or low Aβ42/Aβ40 ratio consistent with AD pathology (see, e.g., Clifford R J, 2018, supra, and references therein, the content of each of which is incorporated herein by reference in its entirety).

According to particular aspects, one or more additional treatment can be administered in combination with the Tau phosphopeptide conjugate. The additional treatment can comprise the administration of a Tau antigen prior to, after or simultaneously with the administration of the conjugate. The antigens in the additional composition need not be identical, but should share antigenic determinants or be substantially similar to the Tau phosphopeptide of the conjugate.

Thus, in certain embodiments, a method of the application further comprises administering to the subject a liposome comprising a Tau phosphopeptide presented on the surface of the liposome. Examples of Tau liposomes useful for the present invention include, but are not limited to, Tau liposomes described in U.S. Pat. Nos. 8,647,631 and 9,687,447, and U.S. patent publication No. US 2019/0119341, the disclose of each is herein incorporated by reference in its entirety. For example, the liposome useful for the application can comprise: a Tau phosphopeptide; a helper T-cell epitope; a lipidated CpG oligonucleotide; and an adjuvant containing a toll-like receptor 4 ligand; wherein the Tau phosphopeptide is presented on the surface of the liposome.

In certain embodiments, administration of an effective amount of a conjugate of the application to a subject results in an anti-pTau IgG or an anti-Tau IgG (non-phosphorylated Tau peptide) response over at least 20 weeks, such as at least 20, 21, 22, 23, 24, 25, 26, 27, 28, 29 or 30 weeks. In other embodiments, administration of an effective amount of a conjugate of the application to a subject results in an IgG response that recognizes pathological ePHF Tau derived from human AD brain, wherein the response lasts over at least 20 weeks, such as at least 20, 21, 22, 23, 24, 25, 26, 27, 28, 29 or 30 weeks.

As used herein, the term “in combination,” in the context of the administration of two or more therapies to a subject, refers to the use of more than one therapy. The use of the term “in combination” does not restrict the order in which therapies are administered to a subject.

The composition can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration.

According to particular embodiments, the kit comprises at least one of a pharmaceutical composition comprising a liposome according to an embodiment of the invention and a pharmaceutical composition comprising a conjugate according to an embodiment of the invention.

### EXAMPLES

### Example 1. Preparation of Conjugate Vaccine

### Peptides and Adjuvants

Tau phosphopeptides (SEQ ID NO: 2) used in this study were produced synthetically (Pepscan, NL) with the phospho-residues added during synthesis. A conjugate comprising a Tau phosphopeptide having the amino acid sequence of SEQ ID NO: 2 covalently linked to a CRM carrier via a linker is herein referred to as JACI-35.054.

Vaccine peptides were conjugated to the carrier protein CRM197 via a polyethylene glycol (PEG)-cysteine-acetamidopropionamide linker. Tau phosphopeptide having the amino acid sequence of SEQ ID NO: 2 was produced synthetically (Polypeptide Laboratories SAS), with phospho-residues and PEG3 spacer added during synthesis. JACI-35.054 was manufactured by conjugating the carrier protein CRM197 via a succinimidyl 3-(bromoacetamide) propionate (SBAP) linker to a cysteine on the N-terminus of the peptide. SBAP was ligated to CRM197 protein primary amines (—NH2) via NHS ester reaction chemistry. The excess SBAP linker was removed using ultrafiltration and diafiltration (UF/DF). The CRM197-SBAP intermediate was conjugated to the Tau phosphopeptide, and once the reaction was completed, the conjugation reaction was terminated by adding excess amount of L-cystine to quench the reaction. The crude CRM197-peptide conjugated product was purified using a Capto Q ImpRes (GE Healthcare) chromatography column and eluted using a salt isocratic method. The purified CRM197-peptide product was then formulated into a buffer containing Tris and sucrose, such as 20 mM Tris, 250 mM Sucrose, at pH 8.1 using UF/DF. The CRM197-tau peptide Drug Substance (DS) stock solution was generated by adding polysorbate 80 (PS80) stock buffer, such as a 10% PS80 stock buffer to reach a final concentration of 0.01% PS80. The solution was thoroughly mixed prior to filtering. Prior to injection, the stock solution was diluted with PBS and CpG/Alum, e.g., to a first concentration of 0.8 mg/mL CRM197-tau peptide, and then further diluted with PBS and CpG/Alum to a final concentration of 30 μg/mL of CRM197-tau peptide for injection. Alternatively, CRM197-tau peptide stock solution was kept at a concentration of 3.1 mg/mL in 10 mM PBS (pH 7.3) and was further diluted in PBS to reach the desired working concentration. CpG oligonucleotide, alum and PBS were then added to reach a final concentration of 30 μg/mL based on CRM197-pTau peptide and the final formulation was thoroughly mixed before injection.

One concern in targeting a CNS antigen with an active vaccine is that non-specific or off-target inflammation might cause unwanted neuropathological changes. To investigate this, whole brains from mice immunized with a conjugate composition were collected and stained to visualize perivascular or other cellular infiltrates. None of the immunized animals had any sign of neuroinflammation, cellular infiltration, or other undesirable neuropathological changes (data not shown). This suggested that the vaccine-induced antibodies, and the innate immune response to vaccination, did not cause neuropathological changes in mice.

### Example 2. Six Month Intramuscular Toxicity Study of JACI-35.054 in Rhesus Monkeys

### Objective

The objective of the whole study was to determine the toxicity of JACI-35.054 (JACI-35.054) following 7 intramuscular (i.m.) injections administered over approximately 6 months to naïve male and female rhesus monkeys and to assess the reversibility of any changes following a 4-week recovery period.

The test and reference items were administered by i.m. injection. Control animals were dosed with the same treatment schedule as per dosed groups receiving compounds deprived of active moieties in the final formulation.

### Design

The study protocol applied was as following (Table 1)

TABLE 1
Protocol for intramuscular toxicity study
of JACI-35.054 in rhesus monkeys.

| | Dose Level | Dose | Number of |
|---|---|---|---|
| Treatment Group | (μg)^{A} | Volume (μL) | Animals |
| 1. Control^{B} | 0 | 700 | 5♂ + 5♀ |
| 2. JACI-35.054 Low Dose^{C} | 15 | 430 | 3♂ + 3♀ |
| 3. JACI-35.054 Mid Dose^{C} | 50 | 500 | 3♂ + 3♀ |
| 4. JACI-35.054 High Dose^{C} | 150 | 700 | 5♂ + 5♀ |

^{A}Doses expressed as CRM197-pTau content.
^{B}Includes tris buffer, 500 μg of CpG7909 and 562.5 μg of aluminum hydroxide suspension/dose.
^{C}Includes CRM197-pTau, 500 μg of CpG7909 and 562.5 μg of alumhydroxide suspension/dose.

The test item JACI-35.054 composition (contains 15, 50, and 150 μg JACI-35.054 at the low, mid, and high dose level, respectively; and 500 μg of CpG7909 and 562.5 μg of aluminum hydroxide suspension) and reference item were administered by intramuscular injection on Days 1, 29, 57, 85, 113, 141 and 169. The control group received the reference item, a combination of tris buffer (instead of active moiety), and CpG7909 and aluminum hydroxide suspensions.

Body weight was assessed on a weekly basis starting from the acclimatization period up the end of the study. Ophthalmoscopy was performed once during pretest and five days after the fourth and last administrations. Electrocardiogram (limb and augmented leads) was recorded for each animal once during the pretest and after the fourth and last administrations. Blood and urine samples were collected for clinical pathology (hematology, coagulation, clinical chemistry and urinalysis) from all animals once during pre-treatment, on Day 90, Day 174 (Main and Recovery) and on all surviving animals on Day 207 (Recovery). Blood samples for serum for determinations of anti-pTau, anti-CRM197 by ELISA were collected on Days −14, 8, 22, 36, 50, 64, 78, 92, 99, 106, 120, 134, 148, 162, 176, 183, (Main and Recovery) and Day 190, 204 and 211 (Recovery). CSF was collected once pre-dose and prior to necropsy. Blood for immunophenotyping was collected pre-dose, on Day 169 and 211 (end of Recovery). PMBCs were collected (ELISpot for T-cell response) on Days −14 and 183 (Main and Recovery) and Day 211 (Recovery). The potential binding activity of the antibodies induced by JACI-35.054 (serum of animals dosed at 150 μg sampled at pretest and on Day 183) to a panel of 42 frozen human tissues from three unrelated individuals was evaluated using immunohistochemistry (IHC). Following the end of the 6-month dosing period and the 4-week recovery period, Main and Recovery animals were euthanized and subjected to necropsy, organ weights and macroscopic observations were recorded. Histopathological examination was performed on the brain, injection sites and lymph nodes of all Main and Recovery study animals.

JACI-35.054 induced anti-pTau IgG titers in all treated monkeys at all tested doses (15, 50 and 150 μg/mL).

### Results

Results from the draft audited report indicate that after seven i.m. injections on Days 1, 29, 57, 85, 113, 141 and 169, all dose levels of JACI-35.054 were well-tolerated by rhesus monkeys with no unexpected mortality, and no JACI-35.054-related clinical signs or effects on body weights, ophthalmology, electrocardiography, immunophenotyping, clinical pathology or cerebrospinal fluid parameters, organ weights or macroscopic/microscopic findings.

There were no notable differences in immune cell population size between study groups for all antibody panels tested. The concentrations of the various cell populations of interest measured in the test-item treated groups (Group 2 to 4) were comparable to control and variations in immune cell population size fell within biological variation for most populations.

Findings observed in some JACI-35.054 treated animals were limited to very slight to moderate skin sensitivity (erythema and edema); however, the transient nature of the findings and absence of persistence or dose relationship did not suggest a trend towards overt skin sensitivity to JACI-35.054. All macroscopic/microscopic changes were considered related to the adjuvant, confounded by experimental procedures and not due to the presence of JACI-35.054 in the injection dose. The slightly lower incidence of microscopic changes and their tendency to be restricted to the muscle layer in the recovery phase may suggest some resolution of the inflammatory and degenerative/necrotic processes following the 4-week recovery period.

The immunohistochemical investigation conducted on 42 frozen human tissues from three unrelated individuals highlighted that the JACI-35.054-induced monkey antibodies, examined at 1/300 and 1/100 dilutions, did not produce off-target staining in any of the tested tissues.

### Conclusion

Overall, due to the absence of apparent test item-related changes after seven intramuscular injections on Days 1, 29, 57, 85, 113, 141 and 169, the highest dose level of JACI-35.054 (150 μg) is considered to be the no observed effect level (NOEL) for this study.

### Example 3. Three Month Repeated Subcutaneous Administration Toxicity Study in Mice

### Objective

The objective of this study was to evaluate the potential toxicity of JACI-35.054 composition, an adjuvanted vaccine formulated with the test item CRM197-pTau and CpG-7909 and aluminum hydroxide as adjuvants, following 7 subcutaneous (SC) injections to CD1 mice over 3 months. On completion of the treatment period, designated animals were euthanized 2 weeks after the last injection (early euthanasia) or after an additional 2-week treatment-free period (late euthanasia) in order to evaluate the reversibility of any findings or potential delayed effects.

### Design

The study protocol applied was as following (Table 2)

TABLE 2
Protocol for subcutaneous administration toxicity study in mice.

| | Dose Level | Dose | Number of |
|---|---|---|---|
| Treatment Group | (μg)^{A} | Volume (μL) | Animals |
| 1. Control^{B} | 0 | 168.3 | 18♂ + 18♀ |
| 2. JACI-35.054 Low Dose^{C} | 1.7 | 141.7 | 12♂ + 12♀ |
| 3. JACI-35.054 Mid Dose^{C} | 5 | 148.3 | 12♂ + 12♀ |
| 4. JACI-35.054 High Dose^{C} | 15 | 168.3 | 18♂ + 18♀ |

^{A}Doses expressed as CRM197-pTau content.
^{B}Includes tris buffer, 50 μg of CpG7909 and 425 μg of aluminum hydroxide suspension/dose.
^{C}Includes CRM197-pTau, 50 μg of CpG7909 and 425 μg of aluminum hydroxide suspension/dose.

The experimental design consisted of Swiss CD1 mice (60 males and 60 females) allocated across 4 groups that were injected SC in the interscapular region (Days 1, 15, 29, 43, 57, 71 and 85) with JACI-35.054 (1.7, 5 and 15 μg/dose; expressed as dose of CRM197-pTau [groups 2, 3, and 4]) or the placebo/control item (Tris buffer, and CpG7909 and aluminum hydroxide adjuvants [group 1]). At the end of the treatment period, 2 weeks after the last administration, the first available 12 animals/sex/group (early euthanasia) were euthanized, while the last 6 animals/sex in groups 1 and 4 (late euthanasia) were euthanized after a 2-week treatment-free period (i.e., 4 weeks after the last test article or control injection). Toxicity parameters and end points evaluated included morbidity/mortality, clinical observations, local injection reactions, body weight, food consumption, ophthalmology, hematology, blood biochemistry and anatomical pathology evaluations (including organ weights). Complete necropsies were performed on all animals with a recording of macroscopic abnormalities for all tissues and microscopic examination (including potential target organs from the groups 1, 2, 3 and 4 animals euthanized at the end of the treatment period and from groups euthanized at the end of the treatment-free period). Blood was collected during pre-treatment, treatment, and treatment-free periods from all animals for determination of immunogenicity as measured by generation of anti-CRM197 IgG and anti-pTau IgG.

### Results

The test item JACI-35.054 (1.7, 5, and 15 μg/dose) and placebo were generally well-tolerated throughout the study and no noteworthy clinical signs or effects on body weight, food consumption, ophthalmology, hematology, or organ weights were observed for the majority of the animals after SC administration. Two animals were found dead during the study (one female in group 1 [control] on Day 78 [Week 12] and one male in group 3 [5 μg] on Day 43 [Week 7]). These mortalities were considered incidental and unrelated to JACI-35.054 administration as one animal belonged to the control group and no unique clinical observations, in-life, or microscopic findings were associated with or identified in the group 3 decedent male. Additionally, no other animals in any JACI-35.054-treated group were found dead or euthanatized for humane reasons during the study.

Following JACI-35.054 administration, anti-pTau IgG titers increased in a generally dose-dependent manner confirming anticipated vaccine-related immunogenicity, and anti-CRM197 IgG titers were induced as well. Additional CRM197-pTau-related changes at the end of treatment included an increased incidence of moderate (grade 3) granulomatous inflammation in males and females receiving 5 μg/dose or above, and slightly increased total protein concentration (+3.6% to +8.1%) and moderately decreased albumin to globulin ratio (A/G; −7.9% to −21.2%) that were considered to reflect a CRM197-pTau-induced increase in globulin concentration due to antigenic stimulation.

Additional noteworthy findings associated with the general SC injection procedure or co-administered adjuvants (particularly the aluminum hydroxide suspension) in all JACI-35.054 treatment groups and the control group included: 1) clinical observations of erythema, thickening and/or swelling at least once during the study (generally observed at a higher frequency and/or severity with a dose-related trend in JACI-35.054-treated animals compared to controls), 2) at necropsy thickening and white masses in the majority of animals that often correlated with microscopic observations of granulomatous inflammation characterized by granulomata with necrotic/caseous centers and/or inflammatory pseudocysts at injection sites, and 3) lymphoid hyperplasia and infiltrates of foamy macrophages in the axillary lymph nodes.

Following the recovery period, the thickening and swelling clinical signs at ≥1.7 μg/animal and granulomatous inflammation at ≥5 μg/animal were fully reversible. Decreased A/G was still observed in females and there was no recovery at the injection sites in any groups. There was partial recovery of adjuvant-related findings in the axillary lymph nodes (decreased incidence/severity or foamy macrophages).

### Conclusion

In conclusion, 7 SC administrations of JACI-35.054 (1.7, 5 and 15 μg/dose) formulated as an adjuvanted vaccine of CRM197-pTau with CpG7909 and alumhydroxide every two weeks to CD1 mice were well-tolerated. Consistent with anticipated immunogenicity, JACI-35.054-related anti-pTau IgG and anti-CRM197 IgG titers were induced in a generally dose-dependent manner. Other CRM197-pTau-related findings were limited to granulomatous inflammation at ≥5 μg/animal/administration that was fully reversible and slightly increased total protein concentration and moderately decreased albumin to globulin ratio that was partially reversible. As all findings were considered to be non-adverse, thus the highest dose level of JACI-35.054 (15 μg) is considered to be the no observed adverse effect level (NOAEL) for this study.

### Example 4. Safety and Efficacy of JACI-35.054 in Humans

A multicenter prospective placebo-controlled, double-blind and randomized study to assess treatment with Tau targeted vaccines versus placebo over 50 weeks (i.e., 12 months) in subjects with early Alzheimer's Disease. The study population is 50-75 years of age (male and female) with a diagnosis of mild AD or MCI due to AD according to National Institute on Aging-Alzheimer's Association (NIA-AA) criteria. Immunizations are performed at months 0 (Week 0), 2 (Week 8), 6 (Week 24) and 12 (Week 48). Based on the safety and immunogenicity results the protocol may be amended to test additional regimens.

### Objectives

Primary Objectives: to assess the safety and tolerability of study vaccines; and to assess the immunogenicity of study vaccines (induction of IgG titers against pTau in serum).

Secondary Objectives: to further assess the immunogenicity of study vaccines (induction of IgG titers against Tau and of IgM titers against pTau and Tau in serum); and to assess the avidity of antibodies elicited by immunization.

Exploratory Objectives: to explore the effect of study vaccines on putative biomarkers of the progression of AD, i.e., blood and/or CSF concentrations of total Tau and pTau proteins; to explore the effect of study vaccines on the activation of T-cells in blood; to explore the activity of study vaccines on blood inflammatory cytokines (e.g., IL-1B, IL-2, IL-6, IL-8, IL-10, IFN-γ, and TNF-α); to further explore the effect of study vaccines on the immune response (e.g., antibodies against vaccine components, functional capacity of vaccine-induced antibodies); and to explore the effect of study vaccines on behavior, cognitive and functional performance.

### Treatment

Up to 3 dose levels of JACI-35.054 administered by the intramuscular route will be tested in up to 3 sub-cohorts. The study currently is ongoing and has been tested in sub-cohort 2.1.

Sub-cohort 2.1 (8 subjects): JACI-35.054 at 15 μg/dose was administered in 6 subjects and the placebo was administered in 2 subjects. The safety and tolerability data after all subjects have received the second injection in sub-cohort 2.1 permit dose escalation after review by Data and Safety Monitoring Board (DSMB).

Sub-cohort 2.2 (8 subjects) (optional): JACI-35.054 at 60 μg/dose are administered in 6 subjects and the placebo is administered in 2 subjects. This sub-cohort is currently being conducted based on good safety and tolerability observed in sub-cohort 2.1 and based on the fact that the antibody response in this previous sub-cohort is anticipated to be optimized at the dose of 60 μg.

Sub-cohort 2.3 (8 subjects) (optional): JACI-35.054 at up to 150 μg/dose may be administered in 6 subjects and the placebo may be administered in 2 subjects. This sub-cohort will be optional and may be conducted based on good safety and tolerability observed in sub-cohort 2.2 and in case the antibody response in this previous sub-cohort is anticipated to be optimized at a higher dose.

Sub-cohort expansion: An optional recruitment extension of up to 16 additional subjects (12 on active treatment and 4 on placebo) may be considered in a given sub-cohort of each cohort. The goal will be to collect additional data at the dose anticipated to present the most favorable profile in terms of immunogenicity, safety and tolerability. The decision to expand a given sub-cohort will be based on accumulated safety/tolerability and immunogenicity data emerging from the respective cohort.

The vaccine or placebo is administered 4 times at respectively weeks 0, 8, 24 and 48, e.g., with 8, 16 and 24 week intervals between each dose. Treatment period is anticipated to be 50 weeks (12 months) and followed by a 24 week (6 months) safety follow-up period. The overall subject participation will be up to around 80 weeks from first screening assessment to last safety follow-up visit.

### Safety Follow-Up

All subjects will be kept under clinical observation for 24 hours after the first administration of study vaccine and for 4 hours after subsequent administrations of study vaccine. A subsequent safety assessment will also be performed 48 to 72 hours after each immunization by telephone call for all study subjects. In each sub-cohort, the first dosing of the first 4 subjects should be performed once the safety assessment at 48 to 72 hours of the previous subject has been performed. Safety laboratory samples will be collected at baseline, prior to each injection and 2-4 weeks after each injection. All treated subjects will have a safety follow-up period of 24 weeks (6 months) after the end of the treatment period. During this period, subjects will be asked to attend a first follow-up visit 19 weeks after the last administration and a last visit at the end of the follow-up period (26 weeks after the last administration). Participants' safety is monitored throughout the study with regular review of safety data by a Data and Safety Monitoring Board (DSMB).

### Interim Analyses (IAs)

Interim analyses of safety, tolerability and immunogenicity data can be conducted in each subcohort as follows:
all subjects in the sub-cohort have completed Visit 4 (Week 10), i.e. 2 to 4 weeks after the second injection—all subjects in the sub-cohort have completed Visit 6 (Week 26), i.e. 2 to 4 weeks after the third injection
all subjects in the sub-cohort have completed Visit 9 (Week 50), i.e. 2 to 4 weeks after the last injection at Week 48
all subjects in the sub-cohort have completed Visit 11 (Week 74), i.e. end of the safety.

### Follow-Up Period

Available biomarker data can also be reviewed during any of these IAs. The IAs described above can also be performed for any expanded sub-cohort.

Additional IAs to review the sustainability of immune response data can be conducted between weeks 26 and 50 and between weeks 50 and 74.

### The Study Population

The study population is 50-75 years of age (male and female) with a diagnosis of mild AD or MCI due to AD according to National Institute on Aging-Alzheimer's Association (NIA-AA) criteria and a Clinical Dementia Rating Scale (CDR) global score of 0.5 or 1.

Inclusion criteria are as follows:
1. Male or female with age from 50 and up to 75 years old inclusive.
2. Mild Cognitive Impairment (MCI) due to AD or Mild AD according to NIA-AA criteria and a Clinical Dementia Rating scale (CDR) global score of 0.5 or 1, respectively.
3. Mini Mental State Examination (MMSE) score of 22 or above.
4. Abnormal level of CSF Abeta amyloid 42 (Aß42) consistent with AD pathology at screening.
   In borderline cases for CSF Aß42 levels, other results may be considered to help determine amyloid positivity e.g., the Aß42/Aß40 ratio and, on a case by case basis, a history of positive amyloid PET scan or positive CSF Aß42 level.
   Results from CSF sampling performed within 6 months prior to screening are acceptable on a case by case basis provided that they are consistent with the presence of amyloid pathology and that the corresponding CSF sample can be used in the study for testing.
5. Subjects either not taking any marketed treatment for AD or receiving a stable dose of an acetylcholinesterase inhibitor and/or memantine for at least 3 months prior to baseline.
6. Subjects cared for by a reliable informant or caregiver to assure compliance, assist with clinical assessments and report safety issues.
7. Women must be post-menopausal for at least one year and/or surgically sterilized. Women of childbearing potential or not post-menopausal must have a negative blood pregnancy test at screening (blood draw between day −14 and day −3 prior to baseline) and be willing to use highly effective methods of contraception from the screening visit until the end of their participation. Urine pregnancy testing will be performed throughout the treatment period to determine if the subject can continue receiving the study vaccine. Male participants in the trial with female partners of child bearing potential are required to use barrier methods of contraception (condoms with spermicide) in addition to contraceptive measures used by female partners during the whole study duration.
8. Subjects who in the opinion of the investigator is able to understand and provide written informed consent.
9. Both subject and informant or caregiver must be fluent in one of the languages of the study and able to comply with all study procedures, including lumbar punctures.

### Exclusion Criteria are as Follows:

1. Participation in previous clinical trials for AD and/or for neurological disorders using active immunization unless there is documented evidence that the subject was treated with placebo only and the placebo vaccine is not expected to induce any specific immune response.
2. Participation in previous clinical trials for AD and/or for neurological disorders using any passive immunization within the past 12 months prior to screening unless there is documented evidence that the subject was treated with placebo only and the placebo is not expected to induce any specific immune response.
3. Participation in previous clinical trials for AD and/or for neurological disorders using any small molecule drug including BACE-1 inhibitors within the past 3 months prior to screening.
4. Concomitant participation in any other clinical trial using experimental or approved medications or therapies.
5. Presence of positive Anti-nuclear Antibody (ANA) titers at a dilution of at least 1:160 in subjects without clinical symptoms of auto-immune disease.
6. Current or past history of auto-immune disease, or clinical symptoms consistent with the presence of auto-immune disease.
7. Immune suppression including but not limited to the use of immunosuppressive drugs or systemic steroids unless they have been prescribed transiently more than 3 months prior to screening.
8. History of severe allergic reaction (e.g., anaphylaxis) including but not limited to severe allergic reaction to previous vaccines and/or medications.
9. Prior history of clinically significant hypoglycaemic episodes.
10. Drug or alcohol abuse or dependence currently met or within the past five years according to Diagnostic and Statistical Manual of Mental Disorders-V (DSM-V) criteria.
11. Any clinically significant medical condition likely to interfere with the evaluation of safety and tolerability of the study treatment and/or the adherence to the full study visit schedule.
12. Any clinically significant medical condition likely to impact the immune system (e.g., any history of acquired or innate immune system disorder).
13. Use of hydralazine, procainamide, quinidine, isoniazide, TNF-inhibitors, minocycline within the last 12 months prior to screening.
14. Use of diltiazem unless on a stable dose for at least 3 months prior to screening.
15. Significant risk of suicide defined, using the Columbia-Suicide Severity Rating Scale, as the subject answering: “yes” to suicidal ideation questions 4 or 5 or answering: “yes” to suicidal behavior within the past 12 months.
16. Concomitant psychiatric or neurologic disorder other than those considered to be related to AD (e.g., head injury with loss of consciousness, symptomatic stroke, Parkinson's disease, severe carotid occlusive disease, TIAs).
17. History or presence of uncontrolled seizures. If history of seizures, they must be well controlled with no occurrence of seizures within 2 years prior to screening. The use of anti-epileptic medications is permitted if at stable dose for at least 3 months prior to screening.
18. History of meningoencephalitis within the past 10 years prior to screening.
19. Subjects with a history of hemorrhagic and/or non-hemorrhagic stroke.
20. Presence or history of peripheral neuropathy.
21. History of inflammatory neurological disorders with potential for CNS involvement.
22. Screening MRI scan showing structural evidence of alternative pathology not consistent with AD which could cause the subject's symptoms. Evidence of space occupying lesions other than benign meningioma of less than 1 cm diameter, more than two lacunar infarcts or one single infarct larger than 1 cm in diameter or any single area of superficial siderosis or evidence of a prior macro-hemorrhage ≥10 mm. Microbleeds on T2* MRI are allowed up to a maximum of 10, regardless of the location.
23. MRI examination cannot be done for any reason, including but not limited to metal implants contraindicated for MRI studies and/or severe claustrophobia.
24. Significant hearing or visual impairment or other issues judged relevant by the investigator preventing to comply with the protocol and to perform the outcome measures.
25. Clinically significant infections or major surgical operation within 3 months prior to screening. Planned surgery anticipated to occur during participation in the study must be reviewed and approved by the medical monitor at screening.
26. Any vaccine received within the past 2 weeks before screening, including influenza vaccine.
27. Clinically significant arrhythmias or other clinically significant abnormalities on ECG at screening.
28. Myocardial infarction within one year prior to baseline, unstable angina pectoris, or significant coronary artery disease.
29. History of cancer within the past 5 years other than treated squamous cell carcinoma, basal cell carcinoma and melanoma in situ, or in-situ prostate cancer or in-situ breast cancer which have been fully removed and are considered cured.
30. Clinically significant deviations from normal values for hematologic parameters, liver function tests, and other biochemical measures, that are judged to be clinically significant in the opinion of the investigator.
31. Pregnancy confirmed by blood test at screening, or subject planning to be pregnant or lactating.
32. Receipt of any anticoagulant drug or antiplatelet drug, except aspirin at doses of 100 mg daily or lower (in order to avoid risk of bleeding during scheduled or unscheduled lumbar puncture).
33. Receipt of any antipsychotic drugs unless on stable low doses for the treatment of insomnia.
34. Donation of blood or blood products within 30 days prior to screening or plans to donate blood while participating in the study.
35. Positive Venereal Disease Research Laboratory (VDRL) consistent with active syphilis at screening.
36. Positive HIV test at screening.
37. Laboratory or clinical evidence of active hepatitis B and/or C at screening.
38. Serum creatinine greater than 1.5× upper limit of normal, abnormal thyroid function tests or clinically significant reduction in serum B12 or folate levels (note: all oral doses of thyroid replacement agents, B12 or folate have to be stable for at least 3 months prior to screening).

### Study Endpoints

The following primary endpoints on safety and tolerability are assessed: adverse events, immediate and delayed reactogenicity (e.g., anaphylaxis, local and systemic reactogenicity, including immune-complex disease); suicidal ideation (C-SSRS); behavior (NPI); cognitive and functional assessments (RBANS, CDR-SB) to assess safety; vital signs; MRI imaging; electrocardiogram; routine hematology and biochemistry evaluation in blood and urine; evaluation of autoimmune antibodies including anti-dsDNA antibodies in blood; inflammatory markers in blood and CSF.

The following primary endpoints on immune response (i.e. immunogenicity) are also assessed: anti-pTau IgG titers in serum (geometric mean, change from baseline, responder rate, peak and area under the curve).

The following secondary endpoints on immune response (i.e. immunogenicity) are assessed: anti-Tau IgG, anti-pTau, anti-ePHF IgG and anti-Tau IgM titers in serum (geometric mean, change from baseline, responder rate, peak and area under the curve), determination of the IgG response profile by avidity testing.

The following exploratory endpoints are assessed: change from baseline of putative AD biomarker titers in blood and/or CSF (e.g., total Tau, pTau), change from baseline in T-cell activation levels as measured in blood, change from baseline of inflammatory cytokine titers in blood, change from baseline in antibody titers in blood, change from baseline in behavior (NPI), cognitive and functional performance (RBANS, CDR-SB) scores.

### Results/Conclusions

The following primary endpoints were/are assessed:
Safety and tolerability—adverse events, immediate and delayed reactogenicity (e.g., anaphylaxis, local and systemic reactogenicity, including pain, redness, immune-complex disease, swelling, fever); global assessment of tolerability; suicidal ideation (C-SSRS); behavior (NPI); cognitive and functional assessments (RBANS, CDR-SB) to assess safety; vital signs; MRI imaging; electrocardiogram; routine hematology and biochemistry evaluation in blood and urine; evaluation of autoimmune antibodies including anti-DNA antibodies in blood; inflammatory markers in blood and CSF.
Immune response—anti-pTau IgG titers in serum (geometric mean, change from baseline, responder rate, peak and area under the curve).

The following secondary endpoints were/are assessed:
Immune response: anti-Tau IgG, anti-pTau, anti-ePHF IgG and anti-Tau IgM titers in serum (geometric mean, change from baseline, responder rate, peak and area under the curve), determination of IgG response profile by avidity testing.

The following exploratory endpoints were/are assessed:
Change from baseline of biomarkers titers in blood and/or CSF (e.g., total Tau and pTau proteins), change from baseline in T-cell activation level in blood, change from baseline of inflammatory cytokine (e.g., IL-1B, IL-2, IL-6, IL-8, IL-10, IFN-γ, and TNF-α) titers in blood, change from baseline in suicidal ideation (C-SSRS), behavior (NPI), cognitive and functional performance (RBANS, CDR-SB) scores.

The study is ongoing. To date, one sub-cohort has received JACI-35.054 at the dosage level of 15 μg of the conjugate (“15 μg dose”), and placebo (phosphate-buffered saline (PBS) solution) as in Table 3. Study subjects in sub-cohort 2.2 are being administered with JACI-35.054 at the dosage level of 60 μg of the conjugate (“60 μg dose”).

TABLE 3
Design of the clinical study.

| | | | Dose | Number of | |
|---|---|---|---|---|---|
| | Sub- | | level ^{A} | early AD | Route of |
| Cohort | cohort | Study treatment | (μg) | subjects # | administration |
| 2 | 2.1 | JACI-35.054^{B} | 15 μg | 6 | Intramuscular |
| | | Placebo (PBS) | 0 μg | 2 | |
| | 2.2 | JACI-35.054^{B} | 60 μg | 6 | Intramuscular |
| | | Placebo (PBS) | 0 μg | 2 | |

^{A} Doses expressed as CRM197-pTau content.
^{B}Includes CRM197-pTau, 500 μg of CpG7909 and 562.5 μg of alum hydroxide suspension/dose.
Dose administered 4 times at following timepoints: Week 0, 8, 24 and 48.
Blood samples for antibody determination withdrawn at following timepoints: Screening, Weeks 0 (pre-dose), 2, 8, 10, 15*, 20*, 24, 26, 31*, 36, 42*, 48, 50, 67 and 74. (*): additional timepoints added; (#) Includes MCI due to AD as well as mild AD subjects
A responder is defined as the number of subjects with an antibody response above a positivity threshold. A post-baseline value is considered positive if ≥ an analytical threshold × baseline antibody titer value. The analytical threshold is defined from samples from human donors (obtained during the validation of each assay).
Baseline antibody titer value is the mean value of the titers measured at screening and visit 1 (including unscheduled visits) provided that they occur prior to the first study vaccine injection.

The interim results up to week 74 (2 weeks after the fourth injection) for the 15 μg dose, and up to week 26 (2 weeks after the third injection) for the 60 μg dose, showed that JACI-35.054 was safe and well tolerated with no clinically-relevant safety concerns related to the study vaccine observed. An increased anti-pTau-specific IgG titer relative to baseline was observed in the serum of the actively treated subjects who were all responders after the second administration of JACI-35.054 at the 15 μg dose at week 10, and also later on at weeks 24, 26, 36, 48, 50, 67 and 74. An increased anti-pTau-specific IgG titer relative to baseline was observed in the serum of the actively treated subjects who were also all responders after the second administration of JACI-35.054 at the 60 μg dose at week 10, and also later on at weeks 15, 20, 24 and 26. An increased anti-Tau-specific IgG titer (response to non-phosphopeptide Tau peptide comprising the amino acid sequence of SEQ ID NO: 4) was also observed in all actively treated study subjects who were all responders at weeks 10, 24, 26, 36, 48, 50, 67 and 74 at the 15 μg dose, and who were all responders at weeks 10, 15, 20, 24 and 26 at the 60 μg dose. Increased anti-ePHF IgG titer to pathological pTau was observed in actively treated early AD subjects, 83.3% of them being responders at weeks 26, 36 and 50 at the 15 μg dose, and 83.3% and 100% of them being responders at weeks 10 and 26, respectively at the 60 μg dose. No antibody response was observed in subjects receiving the placebo.

### Anti-pTau IgG Response of JACI-35.054 in Humans

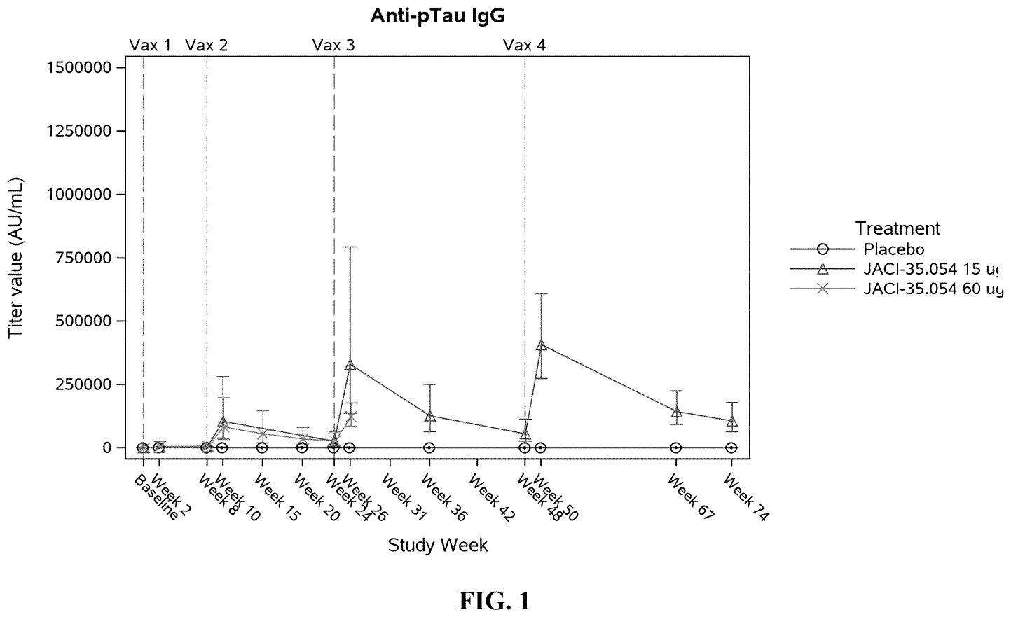

Specific IgG antibody responses directed against a phosphorylated Tau peptide (pTau) induced by JACI-35.054 vaccine in the sub-cohort of Table 3 were measured by MSD. FIG. 1 shows the anti-pTau IgG titers following immunization with either JACI-35.054 at the 15 μg and 60 μg dose, or placebo. As shown by the results in FIG. 1, immunization with JACI-35.054 at either the 15 μg or 60 μg dose induced a potent anti-pTau IgG response directed against a biotinylated phosphorylated tau peptide having the amino acid sequence of SEQ ID NO: 19, which contains biotin linked to the N-terminus of a phosphorylated Tau peptide comprising the amino acid sequence of SEQ ID NO: 2.

Immunizations at weeks 8, 24, and 48 at 15 μg dose level lead to a boosting of the anti-pTau IgG response as measured 2 weeks later, at week 10, week 26, and week 50, respectively. Likewise, immunization at weeks 8 and 24 at 60 μg dose level lead to a boosting of the anti-pTau IgG response as measured 2 weeks later, at weeks 10 and 26, respectively.

Table 4 shows the anti-pTau IgG responder rate (ITT population) following immunization with either JACI-35.054 at the 15 μg dose, 60 μg dose or placebo.

TABLE 4
Anti-pTau IgG responder rate (ITT population) following immunization with
either JACI-35.054 at the 15 μg or 60 μg dose, or placebo.

| | Week | | | | | | | | | | | |
|---|---|---|---|---|---|---|---|---|---|---|---|---|
| | 2 | 8 | 10 | 15 | 20 | 24 | 26 | 36 | 48 | 50 | 67 | 74 |
| JACI-35.054, | 50% | 66.7% | 100% | NA | NA | 100% | 100% | 100% | 100% | 100% | 100% | 100% |
| 15 μg | | | | | | | | | | | | |
| JACI-35.054, | 66.7% | 83.3% | 100% | 100% | 100% | 100% | 100% | NA | NA | NA | NA | NA |
| 60 μg | | | | | | | | | | | | |
| Placebo | 0% | 0% | 0% | 0% | 0% | 0% | 0% | 0% | 0% | 0% | 0% | 0% |

NA = not available

As shown by the results in Table 4, 50% of subjects treated with JACI-35.054 at the 15 μg dose level were responders at week 2, and 66.7% were responders at week 8. All subjects treated with JACI-35.054 at the 15 μg dose level were responders from week 10 up to and including week 74. As shown by the results in Table 4, 66.7% of subjects treated with JACI-35.054 at the 60 μg dose level were responders at week 2, and 83.3% were responders at week 8. All subjects treated with JACI-35.054 at the 60 μg dose level were responders from week 10 up to at least week 26. No subjects treated with placebo generated an anti-pTau IgG response.

### Anti-Tau IgG Response of JACI-35.054 in Humans

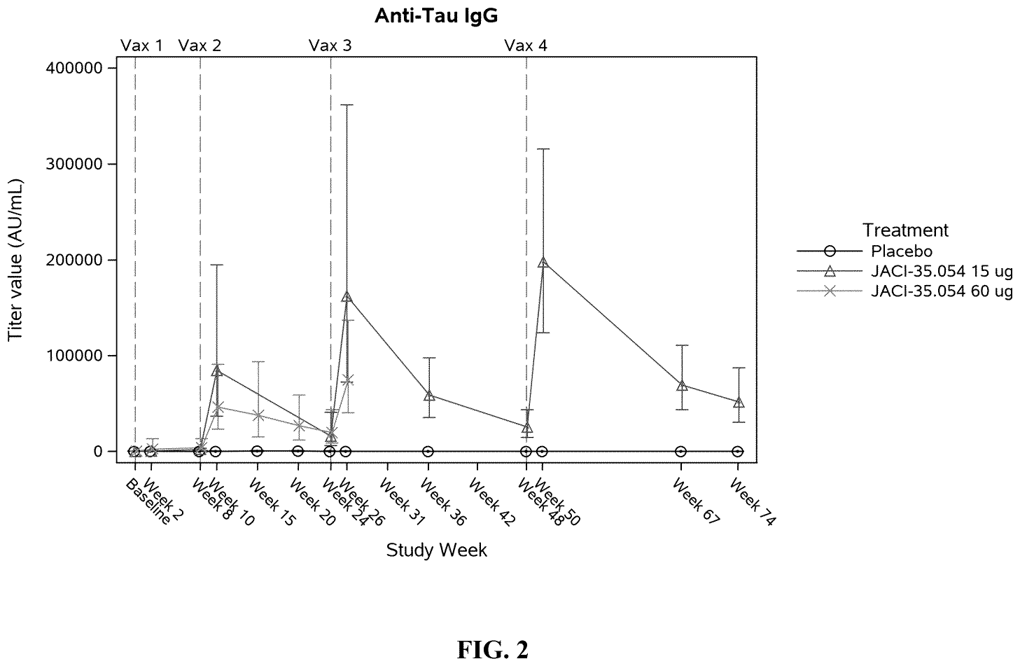

Specific IgG antibody responses directed against a non-phosphorylated Tau peptide induced by JACI-35.054 vaccine in the sub-cohort of Table 3 were measured by MSD. FIG. 2 shows the anti-Tau IgG titers following immunization with either JACI-35.054 at the 15 μg or 60 μg dose or placebo. As shown by the results in FIG. 2, immunization with JACI-35.054 at either the 15 μg or 60 μg dose induced an IgG antibody response directed against a biotinylated non-pTau peptide having the amino acid sequence of SEQ ID NO: 20, which contains biotin linked to the N-terminus of a non-phosphorylated Tau peptide comprising the amino acid sequence of SEQ ID NO: 4. Thus, immunization with JACI-35.054 at 15 μg or 60 μg dose induced an IgG antibody response, which recognizes the non-pTau peptide having the amino acid sequence of SEQ ID NO: 4, in addition to the pTau peptide having the amino acid sequence of SEQ ID NO: 2.

Immunizations at weeks 8, 24, and 48 at the 15 μg dose level led to a boosting of the anti-Tau IgG response as measured 2 weeks later, at weeks 10, 26, and 50, respectively. Immunization at weeks 8 and 24 at the 60 μg dose level led to a boosting of the anti-Tau IgG response as measured 2 weeks later, at weeks 10 and 26, respectively.

Table 5 shows the anti-Tau IgG responder rate (ITT population) following immunization with either JACI-35.054 at the 15 μg or 60 μg dose or placebo. As shown by the results in Table 5, 66.7% of subjects treated with JACI-35.054 at the 15 μg dose level were responders at week 2, and 83.3% were responders at week 8. All subjects treated with JACI-35.054 at the 15 μg dose level were responders from week 10 up to and including week 74. 66.7% of subjects treated with JACI-35.054 at the 60 μg dose level were responders at week 2 and week 8. All subjects treated with JACI-35.054 at the 60 μg dose level were responders from week 10 up to at least week 26. No subjects treated with placebo generated an anti-Tau IgG response.

TABLE 5
Anti-Tau IgG responder rate (ITT population) following immunization with
either JACI-35.054 at the 15 μg or 60 μg dose, or placebo.

| | Week | | | | | | | | | | | |
|---|---|---|---|---|---|---|---|---|---|---|---|---|
| | 2 | 8 | 10 | 15 | 20 | 24 | 26 | 36 | 48 | 50 | 67 | 74 |
| JACI-35.054, | 66.7% | 83.3% | 100% | NA | NA | 100% | 100% | 100% | 100% | 100% | 100% | 100% |
| 15 μg | | | | | | | | | | | | |
| JACI-35.054, | 66.7% | 66.7% | 100% | 100% | 100% | 100% | 100% | NA | NA | NA | NA | NA |
| 60 μg | | | | | | | | | | | | |
| Placebo | 0% | 0% | 0% | 0% | 0% | 0% | 0% | 0% | 0% | 0% | 0% | 0% |

Recognition of Pathological pTau (Enriched Paired Helical Filaments—ePHF) Derived from Human AD Brain

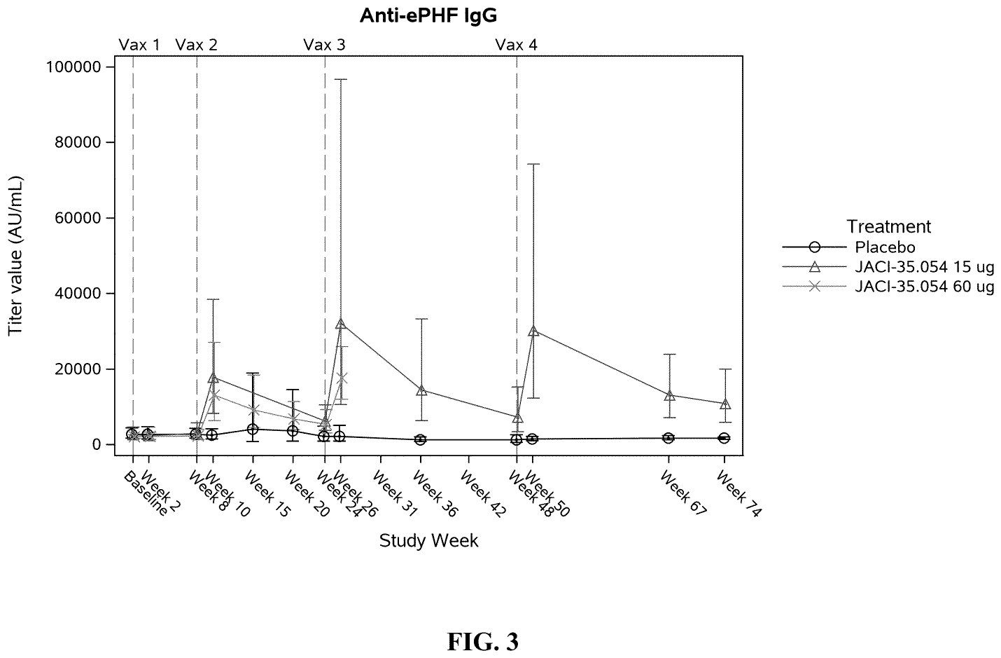

The ability of the IgG polyclonal antibodies induced by immunization with JACI-35.054 in the sub-cohort of Table 3 to bind to ePHF derived from human AD brain was measured over time by MSD. FIG. 3 shows the anti-ePHF IgG titers (ITT population) following immunization with either JACI-35.054 at the 15 μg or 60 μg dose or placebo. The results of FIG. 3 showed that immunization with JACI-35.054 at the 15 μg or 60 μg dose induced an IgG antibody response which recognizes pathological ePHF Tau derived from human AD brain.

Immunizations at weeks 8, 24 and 48 at the 15 μg dose level led to a boosting of the anti-ePHF IgG response as measured 2 weeks later, at weeks 10, 26 and 50, respectively. Likewise, immunization at weeks 8 and 24 at the 60 μg dose level lead to a boosting of the anti-ePHF IgG response as measured 2 weeks later, at weeks 10 and 26, respectively.

Table 6 shows the anti-ePHF IgG responder rate (ITT population) following immunization with either JACI-35.054 at the 15 μg or 60 μg dose level or placebo. Responder rates for anti-ePHF IgG in the 6 subjects treated with JACI-35.054 at the 15 μg dose increased from 0% at week 2 (i.e., 2 weeks after the first injection) to 83.3% at week 26 (i.e., 2 weeks after the 3^{rd} injection), and the responder rate was between 66.7% (week 48, 67 and 74) and 83.3% (week 36 and 50) thereafter. Responder rates for anti-ePHF IgG in the 6 subjects treated with JACI-35.054 at the 60 μg dose increased from 16.7% at week 2 (i.e., 2 weeks after the first injection) to 100% at week 26 (i.e., 2 weeks after the 3^{rd} injection).

TABLE 6
Anti-ePHF IgG responder rate (ITT population) following immunization with
either JACI-35.054 at the 15 μg or 60 μg dose, or placebo.

| | Week | | | | | | | | | | | |
|---|---|---|---|---|---|---|---|---|---|---|---|---|
| | 2 | 8 | 10 | 15 | 20 | 24 | 26 | 36 | 48 | 50 | 67 | 74 |
| JACI-35.054, | 0% | 0% | 66.7% | NA | NA | 50% | 83.3% | 83.3% | 66.7% | 83.3% | 66.7% | 66.7% |
| 15 μg | | | | | | | | | | | | |
| JACI-35.054, | 16.7% | 16.7% | 83.3% | 50% | 50% | 50% | 100% | NA | NA | NA | NA | NA |
| 60 μg | | | | | | | | | | | | |
| Placebo | 0% | 0% | 0% | 0% | 0% | 0% | 0% | 0% | 0% | 0% | 0% | 0% |

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

### Example 5. Vaccination with JACI-35.054 Induces Antibodies with High Heterogeneity in Epitope Recognition

To further profile the antibody response for breadth and selectivity towards pathological pTau, epitope mapping was performed on the human subjects' sera with short pTau and non-pTau amino acid sequences. A study was performed to determine the epitope recognition profile of antibodies induced by JACI-35.054 in human subjects. Eight AD patients were immunized intramuscularly at weeks 0, 8, 24, and 48 with 15 μg per dose of JACI-35.054 or placebo. The epitope recognition profile of antibodies was determined by epitope mapping ELISA before the first immunization (V1, week 0) and after the third immunization (V6, week 26) using a library of N-terminally biotinylated 8-mer peptides, shifted by one amino acid and covering the entire sequence of phospho tau peptide T3.30 (SEQ ID NO: 19) as well as the sequence of tau peptide T3.56 (SEQ ID NO: 20). In addition, binding of antibodies to a full-length phospho tau peptide T3.30 (SEQ ID NO: 19) and tau peptide T3.56 (SEQ ID NO: 20) as well as a phospho tau peptide T3.85 (SEQ ID NO: 21) and tau peptide T3.86 (SEQ ID NO: 22) (with an additional C-terminal amino acid) was determined.

Data is expressed as pre-treatment-subtracted optical density (O.D.) values (O.D. obtained before the initial immunization (V1, week 0) subtracted from O.D. obtained after the third immunization (V6, week 26)) for each peptide and each patient. Negative values after subtraction have been set to 0.000.

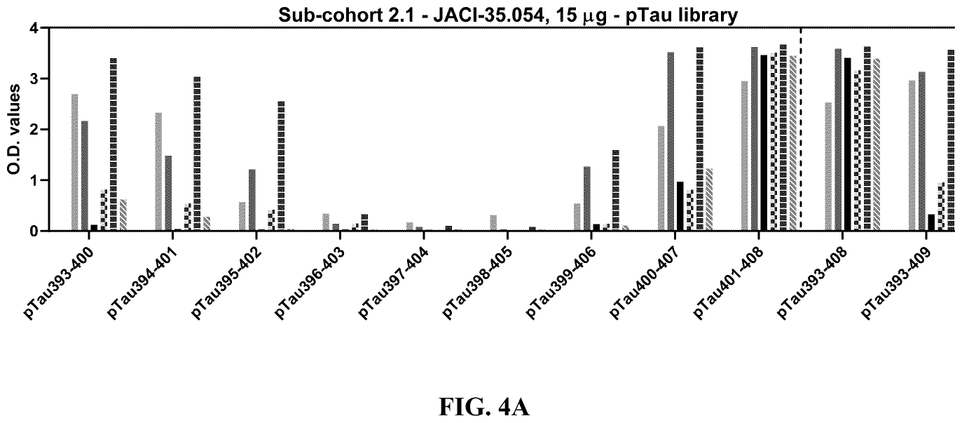

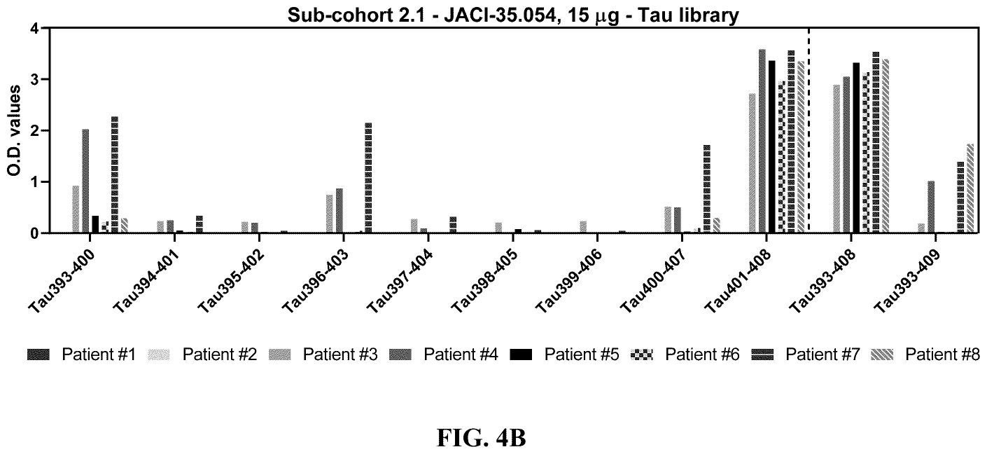

Tables 7 and 8 and FIG. 4 show the epitope recognition profile of antibodies induced by vaccination with JACI-35.054, as determined by epitope mapping ELISA on short 8-mer overlapping peptides, covering phospho-peptide T3.30 (SEQ ID NO: 19) and non-phospho-peptide T3.56 (SEQ ID NO: 20).

TABLE 7

| Phospho-Tau | Patient | | | | | | | |
|---|---|---|---|---|---|---|---|---|
| peptide | #1 | #2 | #3 | #4 | #5 | #6 | #7 | #8 |
| pTau393-400 | 0.000 | 0.000 | 2.694 | 2.162 | 0.119 | 0.815 | 3.402 | 0.622 |
| pTau394-401 | 0.000 | 0.000 | 2.327 | 1.481 | 0.040 | 0.543 | 3.036 | 0.279 |
| pTau395-402 | 0.000 | 0.000 | 0.569 | 1.212 | 0.030 | 0.420 | 2.553 | 0.041 |
| pTau396-403 | 0.000 | 0.000 | 0.342 | 0.145 | 0.028 | 0.147 | 0.327 | 0.025 |
| pTau397-404 | 0.000 | 0.000 | 0.169 | 0.080 | 0.023 | 0.014 | 0.098 | 0.030 |
| pTau398-405 | 0.000 | 0.000 | 0.310 | 0.033 | 0.005 | 0.010 | 0.080 | 0.022 |
| pTau399-406 | 0.000 | 0.000 | 0.545 | 1.269 | 0.138 | 0.146 | 1.591 | 0.109 |
| pTau400-407 | 0.000 | 0.000 | 2.064 | 3.515 | 0.969 | 0.810 | 3.616 | 1.230 |
| pTau401-408 | 0.000 | 0.000 | 2.952 | 3.620 | 3.459 | 3.510 | 3.670 | 3.444 |
| pTau393-408 | 0.003 | 0.000 | 2.531 | 3.584 | 3.405 | 3.162 | 3.624 | 3.395 |
| pTau393-409 | 0.000 | 0.006 | 2.961 | 3.127 | 0.324 | 0.954 | 3.560 | 2.504 |

Table 7 and FIG. 4A show that two AD patients did not produce any IgG antibodies against the sequence of phospho tau peptides T3.30 (SEQ ID NO: 19) and T3.85 (SEQ ID NO: 21) (patients #1 and #2). Six AD patients generated IgG antibodies against the sequence of phospho tau peptide T3.30 (SEQ ID NO: 19) and T3.85 (SEQ ID NO: 21) with overall lower binding to the sequence of phospho tau peptide T3.85 (SEQ ID NO: 21) in at least 4 AD patients. O.D. values obtained on 8-mer peptides indicate that IgG antibodies induced after three immunizations bound to the C-terminal part of the sequence of phospho tau peptide T3.30 (SEQ ID NO: 19) in all six AD patients. In addition, some binding to the N-terminal part of the sequence of phospho tau peptide T3.30 (SEQ ID NO: 19) was found in 4 AD patients.

TABLE 8

| Tau | Patient | | | | | | | |
|---|---|---|---|---|---|---|---|---|
| peptide | #1 | #2 | #3 | #4 | #5 | #6 | #7 | #8 |
| Tau393-400 | 0.013 | 0.000 | 0.927 | 2.026 | 0.337 | 0.228 | 2.271 | 0.287 |
| Tau394-401 | 0.005 | 0.000 | 0.239 | 0.250 | 0.047 | 0.024 | 0.342 | 0.000 |
| Tau395-402 | 0.000 | 0.000 | 0.220 | 0.197 | 0.024 | 0.009 | 0.044 | 0.000 |
| Tau396-403 | 0.000 | 0.000 | 0.745 | 0.871 | 0.017 | 0.042 | 2.151 | 0.004 |
| Tau397-404 | 0.000 | 0.000 | 0.274 | 0.092 | 0.012 | 0.016 | 0.318 | 0.000 |
| Tau398-405 | 0.000 | 0.000 | 0.210 | 0.012 | 0.083 | 0.016 | 0.061 | 0.009 |
| Tau399-406 | 0.000 | 0.000 | 0.234 | 0.010 | 0.006 | 0.017 | 0.042 | 0.002 |
| Tau400-407 | 0.001 | 0.000 | 0.516 | 0.502 | 0.029 | 0.102 | 1.715 | 0.297 |
| Tau401-408 | 0.000 | 0.000 | 2.724 | 3.586 | 3.361 | 2.965 | 3.560 | 3.352 |
| Tau393-408 | 0.000 | 0.000 | 2.890 | 3.053 | 3.326 | 3.141 | 3.535 | 3.387 |
| Tau393-409 | 0.008 | 0.000 | 0.188 | 1.014 | 0.020 | 0.022 | 1.391 | 1.738 |

Table 8 and FIG. 4B shows that two AD patients did not produce any IgG antibodies against the sequence of tau peptides T3.56 (SEQ ID NO: 20) and T3.86 (SEQ ID NO: 22) (patients #1 and #2). Six AD patients generated IgG antibodies against the sequence of tau peptide T3.56 (SEQ ID NO: 20) and T3.86 (SEQ ID NO: 22) with overall lower binding to the sequence of tau peptide T3.86 (SEQ ID NO: 22). O.D. values obtained on 8-mer peptides indicate that IgG antibodies induced after three immunizations bound to the C-terminal part of the sequence of tau peptide T3.56 (SEQ ID NO: 20) in all six AD patients. In addition, binding to the tau peptides tau393-400 and tau396-403 was found in 3 AD patients.

The results indicate that subjects vaccinated with JACI-35.054 demonstrated an antibody response with a strong recognition of the C-terminal part of Tau antigen sequence and the antibodies binding in a similar manner to phosphorylated Tau and non-phosphorylated Tau peptides.

### REFERENCES

Asuni A A et al., J Neurosci. 2007 Aug. 22; 27(34):9115-29
Bentebibel et al., 2013, Sci Transl Med., 5(176):176ra32
Crotty, 2011, Annual Reviews of Immunology. Vol 29: p 621-663
Friedhoff et al., Biochimica et Biophysica Acta 1502 (2000) 122-132
Greenberg and Davies, 1991, Proc Natl Acad Sci USA, 87(15):5827-31
Hanger et al., Trends Mol Med. 15:112-9, 2009
Hickman et al., J. Biol. Chem. vol. 286, N O. 16, pp. 13966-13976, Apr. 22, 2011
Kontsekova E et al., Alzheimers Res Ther. 2014 Aug. 1; 6(4):44
Novak P et al., Lancet Neurology 2017, 16:123-134
Peeraer et al., 2015, Neurobiol Dis., 73:83-95
Ries et al., 2015, Org. Biomol. Chem., 13:9673
Spensieri et al., 2013, Proc Natl Acad Sci USA., 110(35):14330-5
Theunis C et al., PLoS One. 2013; 8(8): e72301
U.S. Pat. No. 7,741,297
U.S. Pat. No. 8,647,631
U.S. Pat. No. 9,687,447
WO90/14837
WO2010/115843

It is claimed:

1. A method of inducing antibodies against Tau, preferably at least one of phosphorylated Tau and enriched paired helical filaments (ePHFs), in a human subject in need thereof, the method comprising administering to the human subject a composition comprising a pharmaceutically acceptable carrier and 5 μg to 200 μg per dose of a conjugate having the structure of formula (I): 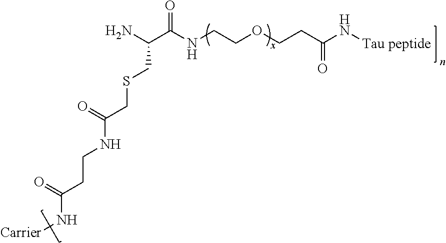
or having the structure of formula (II): 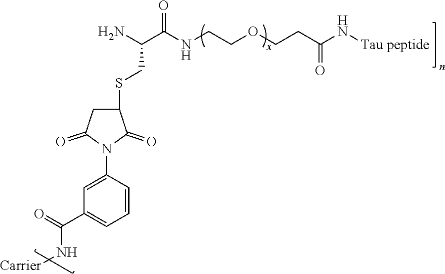
wherein
x is an integer of 0 to 10, preferably 2 to 6, most preferably 3;
n is an integer of 3 to 15, preferably 3 to 12;
Carrier represents an immunogenic carrier selected from the group consisting of keyhole limpet hemocyanin (KLH), tetanus toxoid, CRM197 and an outer membrane protein mixture from N. meningitidis (OMP), or a derivative thereof; and
Tau peptide represents a Tau phosphopeptide having an amino acid sequence selected from the group consisting of SEQ ID NO: 1 to SEQ ID NO: 3 and SEQ ID NO: 5 to SEQ ID NO: 12.

2. The method of claim 1, wherein the Carrier is CRM197.

3. The method of claim 2, wherein the Tau phosphopeptide has the amino acid sequence of SEQ ID NO: 2.

4. The method of claim 3, wherein the conjugate has the structure of: 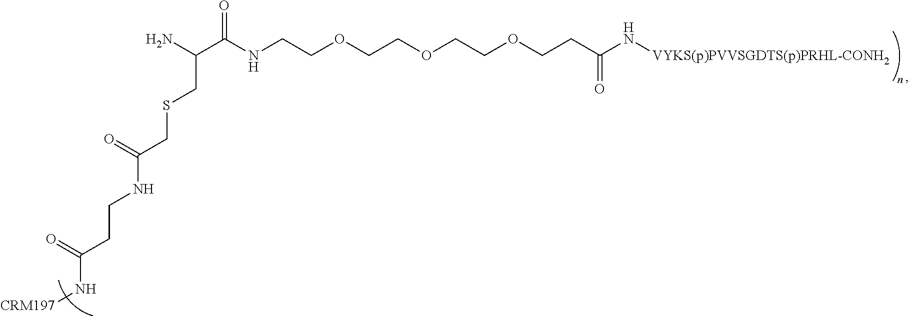
wherein n is an integer of 3 to 7 and VYKS(p)PVVSGDTS(p)PRHL-CONH₂ comprises the phospho-tau peptide of SEQ ID NO:2.

5. The method of any one of claims 1 to 4, wherein the composition further comprises at least one adjuvant.

6. The method of claim 5, wherein the at least one adjuvant comprises a TLR9 agonist.

7. The method of claim 6, wherein the TLR9 agonist is a CpG oligonucleotide having a nucleotide sequence selected from the group consisting of SEQ ID NO: 14 to SEQ ID NO: 18.

8. The method of claim 7, wherein the CpG oligonucleotide has the nucleotide sequence of SEQ ID NO: 14.

9. The method of any one of claims 5 to 8, wherein the at least one adjuvant comprises aluminum hydroxide.

10. A method of inducing antibodies against at least one of phosphorylated Tau and enriched paired helical filaments (ePHFs) in a human subject in need thereof, the method comprising administering to the subject a composition comprising a pharmaceutically acceptable carrier, a CpG oligonucleotide having the nucleotide sequence of SEQ ID NO: 14, and 5 μg to 200 μg per dose of a conjugate having the structure of: 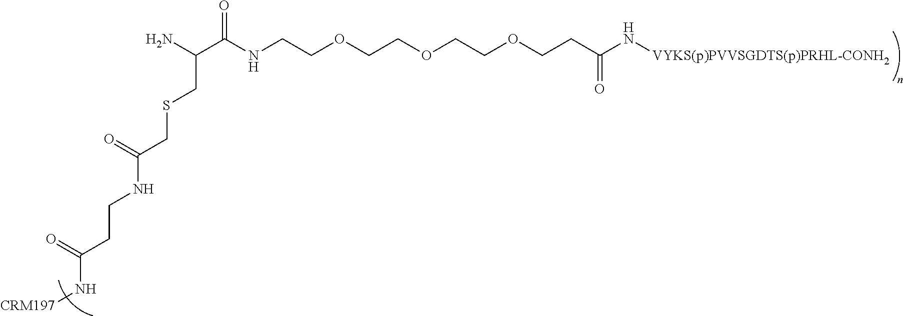
wherein n is an integer of 3 to 7 and VYKS(p)PVVSGDTS(p)PRHL-CONH₂ comprises the phospho-tau peptide of SEQ ID NO:2.

11. The method of claim 10, wherein the composition further comprises aluminum hydroxide.

12. The method of any one of claims 1 to 11, comprising administering to the human subject the composition comprising 5 μg, 10 μg, 15 μg, 20 μg, 25 μg, 30 μg, 35 μg, 40 μg, 45 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, or any value in between, per dose of the conjugate.

13. The method of claim 12, comprising administering to the human subject the composition comprising 15 μg per dose of the conjugate.

14. The method of claim 12, comprising administering to the human subject the composition comprising 45-60 μg, such as 45 μg, 50 μg, 55 μg, 60 μg, or any value in between, per dose of the conjugate.

15. The method of claim 12, comprising administering to the human subject the composition comprising 120-150 μg, such as 120 μg, 125 μg, 130 μg, 135 μg, 140 μg, 145 μg, 150 μg, or any value in between, per dose of the conjugate.

16. The method of any one of claims 1 to 15, wherein the composition is administered intramuscularly.

17. The method of any one of claims 1 to 15, wherein the composition is administered subcutaneously.

18. The method of any one of claims 1-17, wherein the antibodies comprise IgG antibodies against the phosphorylated Tau (pTau), preferably having an anti-pTau IgG titer at least 50, 60, 70, 80, 90, 100 or more times higher than that of a placebo control.

19. The method of any one of claims 1-18, wherein the antibodies comprise IgG antibodies against non-phosphorylated Tau, preferably having an anti-Tau IgG titer at least 50, 60, 70, 80, 90, 100 or more times higher than that of a placebo control.

20. The method of any one of claims 1-19, wherein the antibodies comprise IgG antibodies against an enriched Paired Helical Filament (ePHF), preferably having an anti-ePHF IgG titer at least 2, 3, 4, 5, 6, 7, 8, 9, 10, 11, 12, 13, 14, 15, 16, 17, 18, 19, 20 or more times higher than that of a placebo control.

21. The method of any one of claims 1-20, further comprising administering to the subject a second dose of the composition comprising a pharmaceutically acceptable carrier and 5 μg to 200 μg, such as 15 μg or 60 μg, per dose of the conjugate 4 to 12 weeks, such as 8 weeks, after the initial administration of the composition.

22. The method of claim 21, wherein the administration of the second dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the second dose of the composition.

23. The method of claim 21 or 22, further comprising administering to the subject a third dose of the composition comprising a pharmaceutically acceptable carrier and 5 μg to 200 μg, such as 15 μg or 60 μg, per dose of the conjugate 20 to 28 weeks, such as 24 weeks, after the initial administration of the composition.

24. The method of claim 23, wherein the administration of the third dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the third dose of the composition.

25. The method of claim 23 or 24, further comprising administering to the subject a fourth dose of the composition comprising a pharmaceutically acceptable carrier and 5 μg to 200 μg, such as 15 μg or 60 μg, per dose of the conjugate 44 to 52 weeks, such as 48 weeks, after the initial administration of the composition.

26. The method of claim 25, wherein the administration of the fourth dose of the composition is capable of boosting an antibody response induced by the composition, such as an antibody response comprising an anti-pTau IgG response and/or an anti-ePHF IgG response, preferably the antibody response is increased at least 10%, 20%, 30%, 40%, 50%, 60%, 70%, 80%, 90%, 100% or more, as measured at least 2 weeks after the administration of the fourth dose of the composition.

27. A method of inducing a sustained immune response against a phosphorylated Tau protein (pTau) in a human subject in need thereof, comprising:
i. intramuscularly administering to the subject a primer vaccine comprising an effective amount of a conjugate; and
ii. intramuscularly administering to the subject a first booster vaccine comprising the effective amount of the conjugate 6-10 weeks after the administration of the primer vaccine,
wherein:
the sustained immune response lasts at least about 20 weeks after the administration of the primer vaccine;
the conjugate has the structure of formula (I): 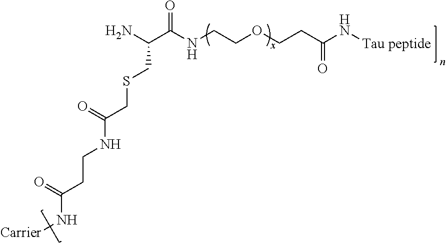
or has the structure of formula (II): 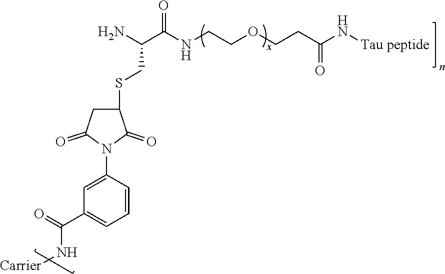
wherein
x is an integer of 0 to 10, preferably 2 to 6, most preferably 3;
n is an integer of 3 to 15, preferably 3 to 12;
Carrier represents an immunogenic carrier selected from the group consisting of keyhole limpet hemocyanin (KLH), tetanus toxoid, CRM197 and an outer membrane protein mixture from N. meningitidis (OMP), or a derivative thereof; and
Tau peptide represents a Tau phosphopeptide having an amino acid sequence selected from the group consisting of SEQ ID NO: 1 to SEQ ID NO: 3 and SEQ ID NO: 5 to SEQ ID NO: 12;
the effective amount of the conjugate comprises 5 μg to 200 μg per dose of the conjugate.

28. The method of claim 27, wherein the Carrier is CRM197.

29. The method of claim 27 or 28, wherein the Tau phosphopeptide has the amino acid sequence of SEQ ID NO: 2.

30. The method of claim 29, wherein the conjugate has the structure of: 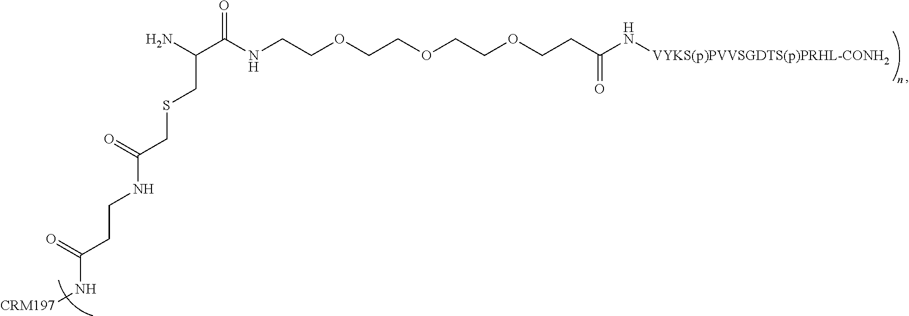
wherein n is an integer of 3 to 7 and VYKS(p)PVVSGDTS(p)PRHL-CONH₂ comprises the phospho-tau peptide of SEQ ID NO:2.

31. The method of any one of claims 27 to 30, wherein the composition further comprises at least one adjuvant.

32. The method of claim 31, wherein the at least one adjuvant comprises a TLR9 agonist.

33. The method of claim 32, wherein the TLR9 agonist is a CpG oligonucleotide having a nucleotide sequence selected from the group consisting of SEQ ID NO: 14 to SEQ ID NO: 18.

34. The method of claim 33, wherein the CpG oligonucleotide has the nucleotide sequence of SEQ ID NO: 14.

35. The method of any one of claims 31 to 34, wherein the at least one adjuvant comprises aluminum hydroxide.

36. The method of any one of claims 27-35, wherein the effective amount of the conjugate comprises 15 μg per dose of the conjugate.

37. The method of any one of claims 27-35, wherein the effective amount of the conjugate comprises 60 μg per dose of the conjugate.

38. The method of any one of claims 27-37, further comprising intramuscularly administering to the subject a second booster vaccine composition comprising the effective amount of the conjugate 20 to 26 weeks after the administration of the primer vaccine, and the sustained immune response lasts at least about 36 weeks after the administration of the primer vaccine.

39. The method of claim 38, wherein the second booster vaccine composition is administered 24 weeks after the administration of the primer vaccine, and the sustained immune response lasts at least about 48 weeks after the administration of the primer vaccine.

40. The method of claim 38 or 39, further comprising intramuscularly administering to the subject a third booster vaccine composition comprising the effective amount of the conjugate 45-50 weeks after the administration of the primer vaccine, and the sustained immune response lasts at least about 67 weeks after the administration of the primer vaccine.

41. The method of claim 40, wherein the third booster vaccine composition is administered 48 weeks after the administration of the primer vaccine, and the sustained immune response lasts at least about 74 weeks after the administration of the primer vaccine.

42. The method of any one of claims 27-41, wherein the sustained immune response comprises an IgG response against phosphorylated Tau (pTau), preferably having an anti-pTau IgG titer at least 50, 60, 70, 80, 90, 100 or more times higher than that of a placebo control.

43. The method of any one of claims 27-42, wherein the sustained immune response comprises an IgG response against non-phosphorylated Tau, preferably having an anti-Tau IgG titer at least 50, 60, 70, 80, 90, 100 or more times higher than that of a placebo control.

44. The method of any one of claims 27-43, wherein the sustained immune response comprises an IgG response against enriched Paired Helical Filament (ePHF), preferably having an anti-ePHF IgG titer at least 2, 3, 4, 5, 6, 7, 8, 9, 10, 11, 12, 13, 14, 15, 16, 17, 18, 19, 20 or more times higher than that of a placebo control.

45. The method of any one of claims 1-44, wherein the subject is in need of clearance of aggregates of Tau.

46. The method of any one of claims 1 to 45, wherein the subject is in need of a treatment of a neurodegenerative disease or disorder caused by or associated with the formation of neurofibrillary lesions.

47. The method of claim 46, wherein the subject is in need of a treatment of Alzheimer's Disease.

48. The method of claim 47, wherein the subject is in need of treatment of an early Alzheimer's Disease or mild cognitive impairment (MCI) due to Alzheimer's Disease.


SEQUENCE LISTING (22 sequences)

SEQ ID NO: 1 - protein, 21 aa, synthetic construct
  SITE 8: note = phosphorylation
  SITE 11: note = phosphorylation
  SITE 14: note = phosphorylation
GDRSGYSSPGSPGTPGSRSRT

SEQ ID NO: 2 - protein, 16 aa, synthetic construct
  SITE 4: note = phosphorylation
  SITE 12: note = phosphorylation
VYKSPVVSGDTSPRHL

SEQ ID NO: 3 - protein, 18 aa, synthetic construct
  SITE 11: note = phosphorylation
  SITE 16: note = phosphorylation
SSTGSIDMVDSPQLATLA

SEQ ID NO: 4 - protein, 16 aa, synthetic construct
VYKSPVVSGDTSPRHL

SEQ ID NO: 5 - protein, 30 aa, synthetic construct
  SITE 18: note = phosphorylation
  SITE 26: note = phosphorylation
RENAKAKTDHGAEIVYKSPVVSGDTSPRHL

SEQ ID NO: 6 - protein, 16 aa, synthetic construct
  SITE 14: note = phosphorylation
RQEFEVMEDHAGTYGL

SEQ ID NO: 7 - protein, 16 aa, synthetic construct
  SITE 7: note = phosphorylation
  SITE 9: note = phosphorylation
PGSRSRTPSLPTPPTR

SEQ ID NO: 8 - protein, 16 aa, synthetic construct
  SITE 7: note = phosphorylation
  SITE 10: note = phosphorylation
GYSSPGSPGTPGSRSR

SEQ ID NO: 9 - protein, 18 aa, synthetic construct
  SITE 4: note = phosphorylation
  SITE 9: note = phosphorylation
GDTSPRHLSNVSSTGSID

SEQ ID NO: 10 - protein, 17 aa, synthetic construct
  SITE 3: note = phosphorylation
  SITE 6: note = phosphorylation
  SITE 13: note = phosphorylation
  SITE 15: note = phosphorylation
PGSPGTPGSRSRTPSLP

SEQ ID NO: 11 - protein, 12 aa, synthetic construct
  SITE 3: note = phosphorylation
HLSNVSSTGSID

SEQ ID NO: 12 - protein, 10 aa, synthetic construct
  SITE 6: note = phosphorylation
VSGDTSPRHL

SEQ ID NO: 13 - protein, 13 aa, synthetic construct
CKDNIKHVPGGGS

SEQ ID NO: 14 - DNA, 24 bp, synthetic construct
  modified_base 1..24: mod_base = OTHER; note = phosphorothioate (ps) internucleotide linkages
  source 1..24: mol_type = other DNA
tcgtcgttttgtcgttttgtcgtt

SEQ ID NO: 15 - DNA, 22 bp, synthetic construct
  modified_base 1..22: mod_base = OTHER; note = phosphorothioate (ps) internucleotide linkages
  source 1..22: mol_type = other DNA
tgactgtgaacgttcgagatga

SEQ ID NO: 16 - DNA, 22 bp, synthetic construct
  modified_base 1..22: mod_base = OTHER; note = phosphorothioate (ps) internucleotide linkages
  source 1..22: mol_type = other DNA
tcgtcgttttcggcgcgcgccg

SEQ ID NO: 17 - DNA, 20 bp, synthetic construct
  modified_base 1..3: mod_base = OTHER; note = phosphorothioate (ps) internucleotide linkages
  modified_base 3..14: mod_base = OTHER; note = phosphodiester (po) internucleotide linkages
  modified_base 14..20: mod_base = OTHER; note = phosphorothioate (ps) internucleotide linkages
  source 1..20: mol_type = other DNA
gggggacgatcgtcgggggg

SEQ ID NO: 18 - DNA, 21 bp, synthetic construct
  modified_base 1..4: mod_base = OTHER; note = phosphorothioate (ps) internucleotide linkages
  modified_base 4..15: mod_base = OTHER; note = phosphodiester (po) internucleotide linkages
  modified_base 15..21: mod_base = OTHER; note = phosphorothioate (ps) internucleotide linkages
  source 1..21: mol_type = other DNA
ggggacgacgtcgtggggggg

SEQ ID NO: 19 - protein, 17 aa, synthetic construct
  SITE 1: note = MISC_FEATURE - biotin linked to the N-terminus
  SITE 5: note = phosphorylation
  SITE 13: note = phosphorylation
GVYKSPVVSGDTSPRHL

SEQ ID NO: 20 - protein, 17 aa, synthetic construct
  SITE 1: note = MISC_FEATURE - biotin linked to the N-terminus
GVYKSPVVSGDTSPRHL

SEQ ID NO: 21 - protein, 17 aa, synthetic construct
  SITE 4: note = phosphorylation
  SITE 12: note = phosphorylation
  SITE 1: note = Biotin-LC linker (Ahx)
VYKSPVVSGDTSPRHLS

SEQ ID NO: 22 - protein, 17 aa, synthetic construct
  SITE 1: note = Biotin-LC linker (Ahx)
VYKSPVVSGDTSPRHLS